(19) United States
(12) Patent Application Publication
(10) Pub. No.: US 2023/0241232 A1
(43) Pub. Date: Aug. 3, 2023

(54) Size-Dependent Brain and Lymphatic Distribution of Macromolecular Drug Delivery Platform

(71) Applicant: The United States of America, As Represented by the Secretary, Department of Health and Human, Bethesda, MD (US)

(72) Inventors: Stephan Timothy Stern, Bethesda, MD (US); David Michael Stevens, Bethesda, MD (US); Jay Arthur Berzofsky, Bethesda, MD (US); Julian Demond Burks, Bethesda, MD (US)

(21) Appl. No.: 18/009,710

(22) PCT Filed: Jun. 9, 2021
(86) PCT No.: PCT/US2021/036548

§ 371 Date: Dec. 9, 2022

Related U.S. Application Data
(60) Provisional application No. 63/037,058, filed on Jun. 10, 2020.

(51) Int. Cl. A61K 47/64

(52) U.S. Cl. CPC A61K 47/645

(57) ABSTRACT

The present invention is directed to a polymer platform comprising poly(L-lysine succinylated) which specifically targets scavenger receptor A1. This platform may be used to conjugate different types of drugs to the polymer for treatment of specific diseases or conditions in a patient. The resulting conjugates display moderate stability or controlled drug release, and allows for delivery and release of drugs and other therapeutic moieties to tissues/cells that express scavenger receptor A1 in a controlled manner.

### CROSS-REFERENCE TO RELATED APPLICATIONS

This application claims priority to U.S. Provisional Application Ser. No. 63/037,058 filed 10 Jun. 2020 and is incorporated herein by reference in its entirety.

### STATEMENT OF GOVERNMENT SUPPORT

This invention was made with Government support under contract number HHSN261200800001E awarded by the National Institutes of Health, National Cancer Institute. The Government has certain rights in the invention.

### BACKGROUND OF THE INVENTION

### 1. Field of the Invention

The present invention is directed to drug delivery platforms, and more specifically to a completely succinylated polymer platform that inherently targets scavenger receptor A1 to deliver drug compounds with great specificity.

### 2. Brief Description of the Related Art

Drug delivery platforms are instruments for selectively delivering a therapeutically active molecular component to target cells. Drug delivery technologies have long claimed the ability to selectively deliver therapeutic cargo to target cells in what is often termed targeted drug delivery. Targeted drug delivery is a method of delivering medication to a patient in a manner that increases the concentration of the medication in some parts of the body relative to others. Ideally, nanomedicine drug delivery platforms would be loaded with drugs and targeted to specific parts of the body where there is solely diseased tissue, thereby avoiding interaction with healthy tissue. The goal of such a system is to target, localize, prolong, and have a protracted drug interaction with the diseased tissue. A targeted system offers several advantages, including reduction in the frequency of the dosages taken by the patient, having a more uniform effects of the drug, reduction of drug side-effects, and reduced fluctuation in drug exposure. However, despite recent breakthroughs in nanomedicine and drug delivery system technology, there is currently no single targeted nanoscale delivery methodology on the market.

Scavenger receptors are cell surface receptors that are structurally diverse, and typically recognize many different ligands to participate in diverse biological functions. The functional mechanisms of scavenger receptors include endocytosis, phagocytosis, adhesion and signaling, and the removal of non-self or altered-self targets. Scavenger Receptor A1 (SR-A1, also known as also known as SCARA1, CD204 or macrophage scavenger receptor 1) was initially identified by its ability to mediate the formation of foam cells, a characteristic component of atherosclerotic lesions. Goldstein J L, Ho Y K, Basu S K, Brown M S (1979) Proc Natl Acad Sci USA 76: 333-337; Kodama T, Freeman M, Rohrer L, Zabrecky J, Matsudaira P, Krieger M (1990) Nature 343: 531-535; Krieger M, Herz J (1994) Annu Rev Biochem 63: 601-637; Bowdish D M, Gordon S (2009) Immunol Rev 227: 19-31. However, more recently, a role beyond the handling of cholesterol in the pathogenesis of cardiovascular diseases has emerged for SR-A1; experiments have shown that SR-A1 not only functions as a phagocytic receptor and an innate immune recognition receptor, but also plays an important role in cell apoptosis and cell proliferation. These receptor characteristics, and myeloid and endothelial expression, make SR-A1 a useful target for treatment of a variety of conditions, such as cancer, infectious disease, and neurodegenerative and inflammatory conditions.

Poly(lysine succinylated) (PLS) polymer has been reported as a potential vehicle for delivery of therapeutically active molecular components. International Patent Application Publication WO94/17829 discloses a method of directing the biodistribution of a small molecule by use of macromolecular polymers in a diagnostic or therapeutic protocol for the treatment of a mammalian recipient. The method includes, among other steps, administering to the recipient a conjugate including a directed biodistribution molecule made from a succinylated polylysine polymer and a diagnostically or therapeutically active small molecule agent, in which the succinyl group is used as a common attachment linker, not a targeting ligand, however, the necessity of free succinate groups to affect distribution is not described. The publication is focused on distribution to renal excretion only, and there is no indication that the directed biodistribution molecule possesses controlled release properties. A prodrug in which a biotin molecule is conjugated to the epsilon (ε)-amino groups of polylysine through an amide group (—C(O)NH—) is disclosed as a specific example. U.S. Pat. No. 6,441,025 to Li et al. discloses water soluble compositions of paclitaxel and docetaxel formed by conjugating the paclitaxel or docetaxel to a water soluble polymer such as poly-glutamic acid, poly-aspartic acid, or polylysine, as well as methods of using the compositions for treatment of tumors, auto-immune disorder, or in coating of implantable stents. However, neither of these references disclose use of poly(lysine succinylated) as a drug delivery platform that targets SR-A1, nor the necessity of available free succinate groups for interaction with SR-A1.

The blood brain barrier is a specialized endothelium that prevents the uptake of substances from the systemic circulation into the central nervous system. This barrier, while protecting the sensitive physiological environment of the brain, is also a major impediment to the treatment of central nervous system (CNS) conditions. A universal drug delivery platform that could distribute drugs to the brain is needed, with wide ranging significance to the fields of psychology, oncology, and infectious and neurodegenerative disease. Development of brain delivery platforms has been one of the longstanding goals of nanomedicine. Although there have been many nanotechnology platforms claiming brain distribution, and several companies developing these platforms are currently in clinical trials, there are no brain delivery platforms that have made it to the market.

What is needed in the art is an improved universal drug delivery platform that can treat diseases and conditions by targeting scavenger receptor A1. Previous literature reported a size-dependent transcytosis of succinylated proteins in brain microvessel endothelial cells (BMECs) in vitro, and these studies suggested that larger proteins with more anionic groups correlated with greater trancytosis in the BMECs (Tokuda H., Masuda S., Takakura Y., Sezaki H., Hashida M., Specific uptake of succinylated proteins via a scavenger receptor-mediated mechanism in cultured brain microvessel endothelial cells. Biochem Biophys Res Commun. 1993, 196 (1):18-24; Tokuda, H.; Nagano, H.; Takakura, Y.; Hashida, M., Uptake Characteristics of Anionized and Cationized Proteins in Brain Microvessel Endothelial Cells. Pharmaceutical Sciences 1997, 3: 147-151). In contrast to these previous findings, the present invention describes a polymeric drug delivery platform, Poly(lysine succinylated) (PLS), which demonstrates greater brain, and lymphatic distribution, for smaller sized polymers not typically utilized for drug delivery. Currently, there are no formulations on the market that effectively increase drug exposure to the brain, and this remains an unmet clinical need. The present invention demonstrates the ability of the PLS platform to undergo a size-dependent receptor-mediated brain uptake, and could act as a novel therapeutic strategy for drug delivery to brain in treatment of brain cancer and neurological disorders.

### SUMMARY OF THE INVENTION

In one aspect, the present invention is directed to a method for delivery of a therapeutically active molecule to the brain or lymphatics of a patient, the method comprising the steps of:
providing a composition comprising a conjugate of completely succinylated poly(lysine succinylated) polymer and a therapeutically active molecule, and
administering the composition to a patient,
wherein said succinylated poly(lysine succinylated) polymer has a molecular weight between 10,000 grams per mole and 25,000 grams per mole,
wherein the conjugate contains free/unmodified succinyl groups available for scavenger receptor A1-targeting;
wherein the conjugate targets scavenger receptor A1;
wherein the conjugate is represented by the following formula

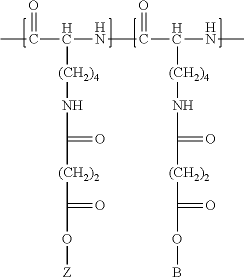

wherein Z=H or Na⁺ and B is a portion of the therapeutically active molecule, and
   wherein the therapeutically active molecule is represented by a general formula B—X, wherein X is OH or NH₂; and wherein the composition targets the brain or lymphatics of the patient.

In another aspect, the present invention is directed to a composition for delivery of a therapeutically active molecule to the brain or lymphatics of a patient, the composition comprising a conjugate of completely succinylated poly(lysine succinylated) and a therapeutically active molecule; wherein the conjugate has a molecular weight between 10,000 and 25,000 grams per mole or greater; wherein the conjugate contains free/unmodified succinyl groups available for scavenger receptor A1-targeting; wherein the conjugate targets scavenger receptor A1; wherein the conjugate is represented by the following formula

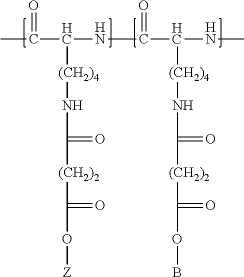

wherein Z=H or Na⁺ and B is a portion of the therapeutically active molecule,
wherein the therapeutically active molecule is represented by a general formula B—X, wherein X is OH or NH₂; and wherein said composition targets the brain and lymphatics of the patient.

These and other aspects of the present invention are described in more detail below.

### BRIEF DESCRIPTION OF THE DRAWINGS

The above and other aspects and features of the present disclosure will become more apparent in the following detailed description when taken in conjunction with reference to the accompanying drawings, in which:

FIG. 1 is a diagram showing a one-step synthesis of polymer-488 using AlexaFluor 488 with poly(lysine succinylated) via EDC HCl and Sulfo-NHS chemistry to form a stable amide bond;

FIG. 2A and FIG. 2B are showing flow cytometry data for untreated and polymer-488 treated RAW 264.7 and clone H cells;

FIG. 3 is a graph of fluorescence (arbitrary units, a. u.) versus concentration of polymer-488 (milligram per milliliter, mg/mL) illustrating fluorescence data from competitive inhibition study;

FIG. 4 is a graph of fluorescence normalized to inhibitor-free control (percent “%” control) versus inhibitor concentration (milligram per milliliter, mg/mL) illustrating fluorescence data from competitive inhibition study, wherein poly I is polyinosinic acid and poly C is polycytidylic acid;

FIG. 5 is a graph of fluorescence (percent “%” control) versus competitor concentration (milligram per milliliter, mg/mL) illustrating fluorescence data from competitive binding study between poly(lysine succinylated) (100% succinylated) and 94% succinylated poly(lysine), wherein polymer-488 excitation/emission is 493/516 nm, data are expressed as mean t SD (n=3), and significance (*) was determined by unpaired t-test p<0.05;

FIG. 6 shows representative whole-body fluorescence images of Balb/c mice treated with Cy7.5 labelled poly(lysine succinylated) via tail vein injection (V=ventral side and D=dorsal side);

FIG. 7 shows representative organ images of Balb/c mice treated with Cy7.5 labelled poly(lysine succinylated) via tail vein injection;

FIG. 8 shows average organ distribution after tail vein injection, wherein the dashed line represents typical background level;

FIG. 9 shows representative whole-body fluorescence images of Balb/c mice treated with Cy7.5 labelled poly(lysine succinylated) via intraperitoneal injection (V=ventral side and D=dorsal side);

FIG. 10 shows representative organ images of Balb/c mice treated with Cy7.5 labelled poly(lysine succinylated) via intraperitoneal injection;

FIG. 11 shows average organ distribution after intraperitoneal injection, wherein the dashed line represents typical background level;

FIG. 12 is a diagram showing anti-alexa-488 staining of fixed tissues following IV or ID administration of polymer-488;

FIG. 13 is a diagram showing non-alcohol-containing drugs conjugated using a multi-step synthesis;

FIG. 14 is a ¹H NMR spectrum of allyl-functionalized poly(L-lysine succinylated) in D20;

FIG. 15 is a diagram showing a one-step synthetic route to conjugate hydroxychloroquine (HCQ) to poly(L-lysine succinylated) through diisopropylcarbodiimide (DIC) coupling;

FIG. 16 shows representative whole-body fluorescence images of Balb/c mice treated with Cy5 labelled poly(lysine succinylated) polymers of molecular weight 10, 25 and 62.5 kDa via tail vein injection;

FIG. 17 shows the average radiance efficiency following ex vivo organ analysis of Balb/c mice treated with Cy5 labelled poly(lysine succinylated) polymers of molecular weight 10 kDa (group 2), 25 kDa (group 3) and 62.5 kDa (group 4) via tail vein injection;

FIG. 18 shows average ratio of lymph node to liver distribution for the Cy5-labelled poly(lysine succinylated) polymers;

FIG. 19A is a graph of peak area to hydroxychloroquine (HCQ) concentration in microgram per milliliter (μg/mL) for the calibration of HCQ standards;

FIG. 19B is a graph of peak area to hydroxychloroquine (HCQ) concentration in microgram per milliliter (μg/mL) for standardization of sodium hydroxide (NaOH)-hydrolyzed HCQ standards;

FIG. 20A shows synthesis of poly(L-lysine succinylated) breflate (PLS-breflate) prodrug via carbodiimide esterification;

FIG. 20B shows NaOH induced ester bond hydrolysis sites for poly(L-lysine succinylated) breflate prodrug;

FIG. 21A shows standard curve of breflate prepared in 50% acetonitrile in water;

FIG. 21B shows a standard curve of NaOH-treated breflate standards;

FIG. 22A is a graph of body weight in grams versus study days illustrating no significant changes in body weight were observed throughout the duration of the study;

FIG. 22B is a graph of tumor volume in cubic millimeters (mm³) versus study days showing the tumor growth for saline control, PLS platform (blank), and PLS-breflate treatment groups throughout the duration of the study. * PLS-breflate treatment groups significantly different from saline and blank PLS controls by ANOVA with fisher's post hoc test (p>0.05).

### DETAILED DESCRIPTION OF THE INVENTION

### Terminology

Compounds are described using standard nomenclature. Unless defined otherwise, all technical and scientific terms used herein have the same meaning as is commonly understood by one of skill in the art to which this invention belongs.

The terms “a” and “an” do not denote a limitation of quantity, but rather denote the presence of at least one of the referenced items. The term “or” means “and/or”. The terms “comprising,” “having,” “including,” and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein may include heavy or radioactive isotopes in the structure of the compounds or as substituents attached thereto. Examples of useful heavy or radioactive isotopes include ¹⁸F, ¹⁵N, ¹⁸O, ⁷⁶Br, ¹²⁵I and ¹³¹I.

The opened ended term “comprising” includes the intermediate and closed terms “consisting essentially of” and “consisting of.”

A dash (“—”) that is not between two letters or symbols is used to indicate a point of attachment for a substituent.

“Conjugate” means a chemical entity, in which two or more compounds are bonded to each other through a coordination, covalent, or ionic bond.

“Pharmaceutical compositions” means compositions comprising at least one active agent, such as a compound or salt of Formula 3, and at least one other substance, such as a carrier. Pharmaceutical compositions meet the U.S. FDA's GMP (good manufacturing practice) standards for human or non-human drugs.

A “patient” means a human or non-human animal in need of medical treatment. Medical treatment can include treatment of an existing condition, such as a disease or disorder or diagnostic treatment. In some embodiments the patient is a human patient.

“Providing” means giving, administering, selling, distributing, transferring (for profit or not), manufacturing, compounding, or dispensing.

“Treatment” or “treating” means providing an active compound to a patient in an amount sufficient to measurably reduce any disease symptom, slow disease progression or cause disease regression. In certain embodiments treatment of the disease may be commenced before the patient presents symptoms of the disease.

A “physiologically effective amount” of a pharmaceutical composition means an amount effective, when administered to a patient, to provide a therapeutic benefit such as an amelioration of symptoms, decrease disease progression, or cause disease regression.

A “therapeutically active molecule” means a compound which can be used for diagnosis or treatment of a disease. The compounds can be small molecules, peptides, proteins, or other kinds of molecules.

A significant change is any detectable change that is statistically significant in a standard parametric test of statistical significance such as Student's T-test, where p<0.05.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within t 30%, 20%, 10% or 5% of the stated value.

### Embodiments

As indicated above, one embodiment of the invention is directed to a method for delivery of a therapeutically active molecule to a patient through targeting scavenger A1 receptor is provided, smaller molecular weight poly(L-lysine succinylated) polymers [3000 (3 k) and 35000 (35 k)] achieved a greater distribution to brain and lymphatics in comparison to a larger, 62,500 (62.5 k) polymer based on an in vivo biodistribution study in mice. For example, the molecular weight of poly(L-lysine succinylated) polymers is about or in any range between about 3000, 5000, 7000, 10000, 13000, 15000, 17000, 19000, 21000, 23000, 25000, 27000, 29000, 31000, 33000, and 35000 grams per mole, for example, between about 3000 grams per mole to about 35000 grams per mole, about 5000 grams per mole to about 30000 grams per mole, or about 10000 grams per mole to about 25000 grams per mole. In an embodiment, the molecular weight of poly(L-lysine succinylated) polymers is between 10000 grams per mole and 25000 grams per mole. The method includes the steps of providing a composition including a conjugate of poly(lysine succinylated) and a therapeutically active molecule, and administering the composition to a patient, wherein the conjugate displays affinity for scavenger A1 receptor. In another embodiment, a composition for delivery of a therapeutically active molecule by way of targeting to scavenger A1 receptor to a patient is provided. The composition includes a conjugate of poly(lysine succinylated) and a therapeutically active molecule. These embodiments are described in more detail below.

In one embodiment, the present invention is directed to a succinylated polymer conjugate that inherently targets scavenger receptor A1 to deliver drug compounds with great control and specificity, and a method of delivering therapeutically active molecules to specific targets in a patient using the succinylated polymer conjugate. It demonstrates the ability of the poly(L-lysine succinylated) (PLS) platform to undergo a size-dependent receptor-mediated brain and lymphatic uptake. The conjugate is based on the anionic polymer poly(L-lysine succinylated), which itself displays high affinity for the scavenger receptor A1 and does not require attachment of any ligands specifically targeting the receptor. The conjugate includes a succinyl moiety bonded to the ε-amino group of L-lysine, wherein the succinyl moiety includes a pendant carboxylic acid group capable of conjugating to a drug molecule through a hydrolyzable ester bond or stable amide bond. As will be discussed below, various drug molecules may be attached to the carboxylic acid group of poly(L-lysine succinylated) to form a poly(L-lysine succinylated) conjugate. A poly(L-lysine succinylated) conjugate, as used herein, is therefore defined as a chemical entity in which a therapeutically active molecule is bonded to the poly(L-lysine succinylated) through an ester bond or amide bond. Such a poly(L-lysine succinylated) conjugate may find utility in a variety of applications including drug delivery to or imaging of the tissues expressing scavenger receptor A1 (such as liver and lymphatics), treatment of lymphoid/macrophage HIV reservoirs, and targeting of tumor associated macrophage, among others. Each of the components of the poly(L-lysine succinylated) conjugate is described in more detail below.

As used herein, the term “poly(lysine succinylated)” refers to a polymer having the following structure:

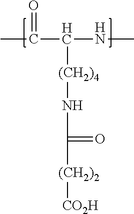

Poly(lysine succinylated) may be prepared, for example, by succinylation of poly-L-lysine with succinic anhydride in the presence of a base. As a result of the reaction, some or all of the primary amino groups become succinylated, including terminal and ε-amino groups. Succinylation of some of the amino groups of poly-L-lysine results in a partially succinylated poly-L-lysine. Succinylation of all or substantially all amino groups of poly-L-lysine provides a completely succinylated poly-L-lysine. As used herein, the term “succinylation of substantially all amino groups” refers to succinylation of amino groups, present in poly-L-lysine, in an amount of 99% or greater, for example, 99.5% or greater, or 99.9% or greater. Therefore, the degree of succinylation in the completely succinylated poly-L-lysine may be 99% or greater, for example, 99.5% or greater, or 99.9% or greater. Remarkably, it was observed that smaller molecular weight poly(L-lysine succinylated) polymers (10 k and 25 k) achieved greater distribution to brain and lymphatics in comparison to a larger, 62.5 k polymer based on an in vivo biodistribution study in mice.

The molecules of poly(lysine succinylated) include carboxylic acid groups, which are capable of reacting with compounds having hydroxyl groups, such as alcohols or phenols, to produce hydrolysable esters, or amines to form stable amide bonds. Accordingly, various hydroxyl containing molecules B—OH can be attached by way of an ester linkage to poly(lysine succinylated) to form a conjugate. The attachment may be schematically represented as follows:

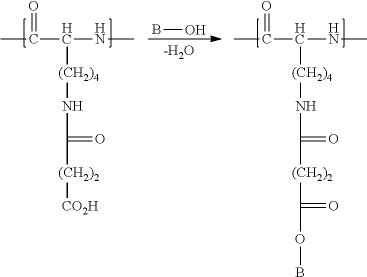

In some other embodiments, various amine containing molecules B—NH₂ can be attached by way of an amide linkage to poly(lysine succinylated) to form a conjugate. The attachment may be schematically represented as follows:

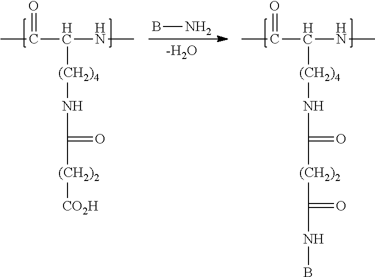

In an embodiment, B—OH or B—NH₂ may be a therapeutically active molecule capable of producing a biological effect. For example, the therapeutically active molecule may be a drug molecule useful for treatment of a disease or condition selected from acne, attention deficit/hyperactivity disorder (ADHD), human immunodeficiency virus (HIV), Rift Valley fever virus, allergies, Alzheimer's disease, angina, anxiety, arthritis, asthma, bipolar disorder, bronchitis, cancer, cirrhosis, elevated cholesterol problems, cold and flu, constipation, chronic obstructive pulmonary disease (COPD), depression, type 1 and 2 diabetes, diarrhea, eczema, erectile dysfunction, fibromyalgia, gastrointestinal disorders, gastroesophageal reflux disease (GERD), gout, hair loss, hay fever, heart disease, hepatitis A, hepatitis B, hepatitis C, hypertension, hypothyroidism, incontinence, inflammatory conditions, irritable bowel syndrome, insomnia, menopause, mental health, migraine, obesity, osteoarthritis, osteoporosis, pain, psoriasis, rheumatoid arthritis, schizophrenia, seizures, sexually transmitted disorder (STD), stroke, swine flu, urinary tract infection (UTI), weight loss, but are not limited thereto.

In an embodiment, the hydroxyl group containing molecule B—OH or the amine group containing molecule B—NH₂ may be a small molecule drug, a peptide, or a vaccine. The inventors of the present invention have found that the poly(L-lysine succinylated) may conjugate different types of drugs or other moieties to the polymer to achieve a moderately stable (i.e., controlled) release of the therapeutic component. Because of its high affinity for scavenger receptor A1, poly(L-lysine succinylated) may thus serve as a convenient platform to deliver various therapeutically active molecules to tissues/cells that express scavenger receptor A1.

In a preferred embodiment, the therapeutically active molecule may be an anti-cancer drug such as PI-103, or brefeldin A or its prodrug breflate, an immunotherapy drug such as galactosyl ceramide, an anti-viral drug such as lamivudine, an anti-parasitic drug such as hydroxychloroquine, a CNS drug such as trehalose, an antibacterial drug such as rifampicin, or a combination of any of these.

Examples of useful anti-cancer drugs useful in the invention include gemcitabine, rapamycin, PI-103, PF-04691502, AZD-8055, torkinib, KU-0063794, PX-886, apitolisib, everolimus, hydroxychloroquine, resiquimod, glactosylceramide, or breflate. Examples of useful anti-viral drugs include abacavir, atazanavir, everolimus, lamivudine, emtricitabine, lopinavir, rapamycin, ritonavir, tenofovir, dolutegravir, zidovudine, hydroxychloroquine, or ribavarin. Examples of useful anti-parasitic drugs include hydroxychloroquine, quinine, mefloquine, doxycycline, atovaquone, metronidazole, or ivermectin. Examples of useful CNS drugs include valproate, haloperidol, galantamine, gabapentin, rotigotine or trehalose. PI-103, hydroxychloroquine, trehalose, and lamivudine are particularly suitable for the compounds and methods of the present invention because each contain hydroxyl groups that may be attached to poly(L-lysine succinylated) through an ester linkage —C(═O)O—. Specific examples of therapeutic formulations, including PI-103 (as a model chemotherapeutic) and lamivudine (as a model anti-HIV drug), have been developed and are described below. The prodrugs showed specificity of almost 100% to the macrophage cell lines containing the scavenger A1 receptor. Other examples of suitable therapeutic compounds useful in the present invention may include gemcitabine (as another model chemotherapeutic), rapamycin (as an anti-viral or anti-cancer drug), everolimus (as an analog of rapamycin), breflate (as a water soluble prodrug for an antineoplastic agent brefeldin A), and galactosylceramide (as an immunomodulatory agent).

The amount of the therapeutically active molecule B—OH or —NH₂ in the poly(lysine succinylated) conjugate may be about 1% or greater based on the total weight of the poly(lysine succinylated) conjugate. For example, the amount of the therapeutically active molecule in the poly(lysine succinylated) conjugate may be about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, or about 75%, or greater, based on the total weight of the conjugate. For example, the amount of the therapeutically active molecule in the poly(lysine succinylated) conjugate may be about 1% to about 5%, about 1% to about 10%, about 1% to about 20%, about 1% to about 25%, about 1% to about 30%, about 1% to about 35%, about 1% to about 40%, about 1% to about 45%, about 1% to about 50%, about 1% to about 55%, about 1% to about 60%, about 1% to about 65%, about 1% to about 70%, about 1% to about 75%, or greater, based on the total weight of the conjugate.

The number of the therapeutically active molecules conjugated per molecule of poly(lysine succinylated) may be about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, or greater. For example, the number of the therapeutically active molecules conjugated per molecule of poly(lysine succinylated) may be about 1-10, about 2-20, about 3-30, about 4-40, about 5-50, about 10-20, about 10-30, about 10-40, about 10-50, about 10-60, about 10-70, about 20-30, about 20-40, about 20-40, about 20-50, about 20-60, about 20-70, or greater.

The amount of the completely succinylated poly(lysine succinylated) polymer portion in the conjugate may be about 25% or greater based on the total weight of the conjugate. For example, the amount of the completely succinylated poly(lysine succinylated) polymer portion in the conjugate may be about 25%, about 30%, about 35%, about 40%, about 45%, about 50%, about 55%, about 60%, about 65%, about 70%, about 75%, about 80%, about 90%, or about 95%, or greater, based on the total weight of the conjugate. For example, the amount of the completely poly(lysine succinylated) polymer portion in the conjugate may be about 25-30%, about 25-35%, about 25-40%, about 25-45%, about 25-50%, about 25-55%, about 25-60%, about 25-65%, about 25-70%, about 25-75%, about 25-80%, about 25-85%, about 25-90%, about 25-95%, or greater, based on the total weight of the conjugate.

As noted above, poly(L-lysine succinylated) may be either partially or completely succinylated. A complete succinylation results in substantially 100% conversion of all primary amino groups to succinate groups which are necessary for conjugation of drugs through esterification. Because the succinylated groups also act as targeting ligands, a complete succinylation of the poly-L-lysine offers a number of advantages such as increased targeting of scavenger receptor A1 and maximization of drug loading. The complete succinylation provides the maximum number of succinylated sites on the polymer, which allows for high drug loading while still having available pendant succinate groups that are necessary for targeting scavenger receptor A1. In contrast, a partial succinylation results in less than 100% conversion of all primary amines to succinate groups, with unmodified amino groups being present in the polymer. Since the unmodified amino groups may interfere with subsequent conjugation reactions of the drug to the polymer, they must be protected by a reaction with a capping agent, such as acetic anhydride. Thus, the use of a partially succinylated poly-L-lysine results in a decreased number of succinylated sites on the polymer, reduced targeting capacity, and decreased drug loading.

The composition including a conjugate, according to an embodiment of the present invention, has controlled drug release properties. Most formulations known in the prior art (prodrugs, micelles, nanoparticles, liposomes) are either very stable (i.e., release the drug too slowly to achieve efficacy) or unstable (i.e., release most or all drug immediately or within an hour of dilution in plasma). With regard to the prior art formulations, it is not uncommon to use the term “controlled release” or similar phrases. However, more often than not, researchers are evaluating drug release formulations in vitro using either non-optimal conditions or non-physiological media. Most drug release assays reported in the prior art use phosphate-buffered saline (PBS) as a release media. Nonetheless, the prior art formulations that appear to be stable and release the drug slowly in PBS often, dissociate immediately when placed into plasma. In contrast, the inventors of the present invention discovered new poly(L-lysine succinylated) prodrugs generally having a drug release half-life of about 3-80 hours, for example, 10-50 hours in plasma.

While the scavenger receptor A1 has a number of reported ligands, to prepare a prodrug, most research groups use a known ligand or inhibitor of the receptor to conjugate to a nanoparticle or polymer in order to increase affinity of the formulation for the receptor. In contrast, the poly(L-lysine succinylated) prodrug, according to an embodiment of the present invention, shows itself high affinity for the receptor through succinylated amino-groups, and does not need to be conjugated to any additional targeting ligands.

The conjugates, according to an embodiment of the present invention, also display remarkable 100% specificity for cells that express scavenger receptor A1, after 24 hours of incubation. While there are multiple mechanisms for particles/formulations to be taken up by cell during this substantial period, it is surprising to see that the polymer does not bind at all to the cells that do not express scavenger receptor A1.

In an embodiment, the poly(lysine succinylated) PI-103 conjugate has the following formula:

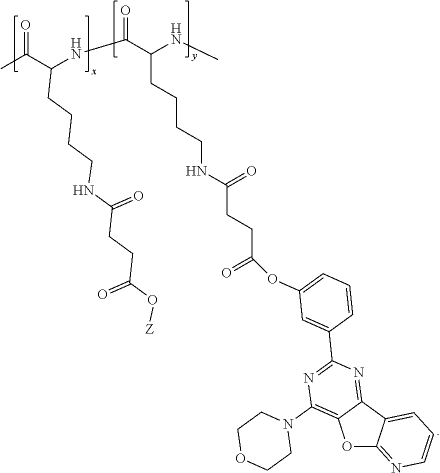

In the above formulae, x and y may be variables selected such that x+y=1, and Z may be H or Na. In the above formula, “x” and “y” represent molar fractions of the corresponding repeating units constituting the conjugate, and “x+y=1” means that the conjugate essentially includes repeating units designated by “x” and “y”, and does not include any other repeating units in substantial quantity (the sum of the molar fractions of the repeating units designated by “x” and “y” adds up to constitute a whole, which is “1”).

For example, y may be an integer between 1 and 10, and x may be (40-y) or (100-y), depending on the length of the polymer.

In an embodiment, a composition including a conjugate of poly(L-lysine succinylated) and lamivudine is provided. The conjugate has the following formula:

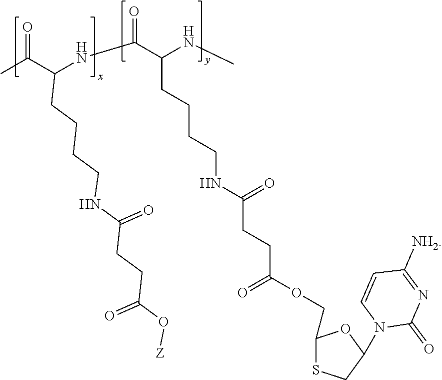

In the above formula, x and y may be variables selected such that x+y=1, and Z may be H or Na. For example, y may be an integer between 1 and 10, and x may be (40-y) or (100-y), depending on the length of the polymer.

In another embodiment, a composition including a conjugate of poly(L-lysine succinylated) and emtricitabine is provided. The conjugate has the following formula:

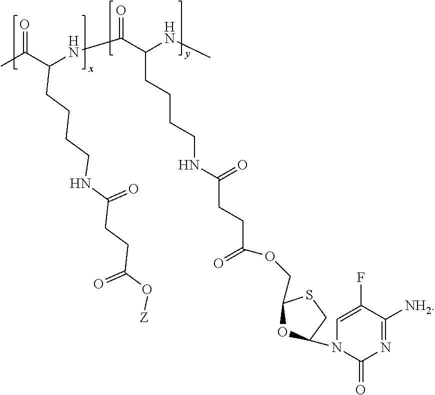

In the above formula, x and y may be variables selected such that x+y=1, and Z may be H or Na.

For example, y may be an integer between 1 and 10, and x may be (40-y) or (100-y), depending on the length of the polymer.

In another embodiment, a composition including a conjugate of poly(L-lysine succinylated) and hydroxychloroquine (HCQ) is provided. The conjugate has the following formula:

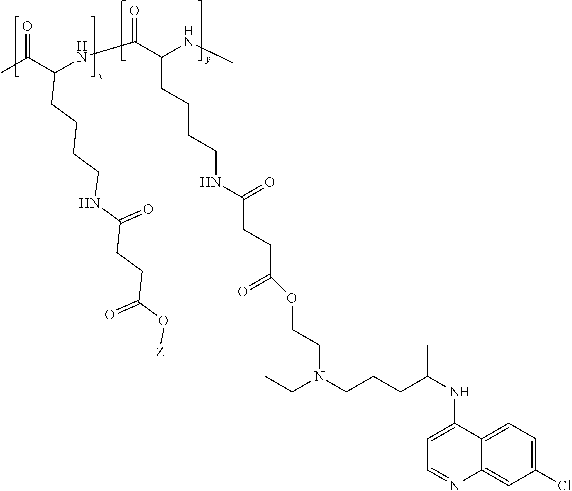

In the above formula, x and y may be variables selected such that x+y=1, and Z may be H or Na. For example, y may be an integer between 1 and 10, and x may be (40-y) or (100-y), depending on the length of the polymer.

In another embodiment, a composition including a conjugate of poly(L-lysine succinylated) and trehalose is provided. The conjugate has the following formula:

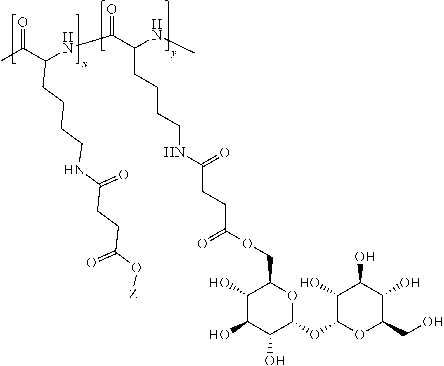

In the above formula, x and y may be variables selected such that x+y=1, and Z may be H or Na. For example, y may be an integer between 1 and 10, and x may be (40-y) or (100-y), depending on the length of the polymer.

In another embodiment, a composition including a conjugate of poly(L-lysine succinylated) and breflate is provided. The conjugate has the following formula:

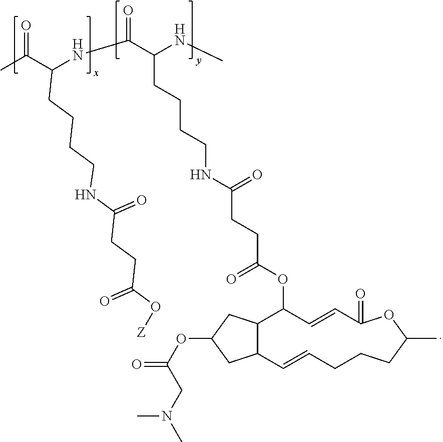

In the above formula, x and y may be variables selected such that x+y=1, and Z may be H or Na. For example, y may be an integer between 1 and 10, and x may be (40-y) or (100-y), depending on the length of the polymer.

The composition may further include at least one pharmaceutically acceptable excipient. A pharmaceutically acceptable excipient, as used herein, refers to a non-active pharmaceutical ingredient (“API”) substance such as a disintegrator, a binder, a filler, and a lubricant used in formulating pharmaceutical products. Each of these substances is generally safe for administering to humans according to established governmental standards, including those promulgated by the United States Food and Drug Administration (“FDA”).

A disintegrator, as used herein, refers to one or more of agar-agar, algins, calcium carbonate, carboxymethylcellulose, cellulose, clays, colloid silicon dioxide, croscarmellose sodium, crospovidone, gums, magnesium aluminium silicate, methylcellulose, polacrilin potassium, sodium alginate, low substituted hydroxypropylcellulose, and cross-linked polyvinylpyrrolidone hydroxypropylcellulose, sodium starch glycolate, and starch, but is not limited thereto.

A binder, as used herein, refers to one or more of microcrystalline cellulose, hydroxymethyl cellulose, and hydroxypropylcellulose, but is not limited thereto.

A filler, as used herein, refers to one or more of calcium carbonate, calcium phosphate, dibasic calcium phosphate, tribasic calcium sulfate, calcium carboxymethylcellulose, cellulose, dextrin derivatives, dextrin, dextrose, fructose, lactitol, lactose, magnesium carbonate, magnesium oxide, maltitol, maltodextrins, maltose, sorbitol, starch, sucrose, sugar, and xylitol, but is not limited thereto.

A lubricant, as used herein, refers to one or more of agar, calcium stearate, ethyl oleate, ethyl laureate, glycerin, glyceryl palmitostearate, hydrogenated vegetable oil, magnesium oxide, magnesium stearate, mannitol, poloxamer, glycols, sodium benzoate, sodium lauryl sulfate, sodium stearyl, sorbitol, stearic acid, talc, and zinc stearate, but is not limited thereto.

In an embodiment, a method for delivery of a therapeutically active molecule to a patient through targeting scavenger A1 receptor is provided. The method includes the steps of providing a composition including a conjugate of poly(lysine succinylated) and a therapeutically active molecule, as described above, and administering the composition to a patient.

The composition according to the present invention may be administered to a patient by various routes. Examples of routes of administration include, but are not limited to, parenteral, e.g., intravenous, intradermal, subcutaneous, oral, intranasal (e.g., inhalation), transdermal (e.g., topical), transmucosal, and rectal administration. In an embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer.

In accordance with any of the embodiments, the composition according to the present invention can be administered orally to a subject in need thereof. Formulations suitable for oral administration can consist of (a) liquid solutions, such as an effective amount of the compound dissolved in diluents, such as water, saline, or orange juice and include an additive, such as cyclodextrin (e.g., α-, β-, or γ-cyclodextrin, hydroxypropyl cyclodextrin) or polyethylene glycol (e.g., PEG400); (b) capsules, sachets, tablets, lozenges, and troches, each containing a predetermined amount of the active ingredient, as solids or granules; (c) powders; (d) suspensions in an appropriate liquid; and (e) suitable emulsions and gels. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The composition according to the present invention can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations will typically contain from about 0.5 to about 25% by weight of the composition according to the present invention in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The composition according to the present invention may be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Topically applied compositions are generally in the form of liquids (e.g., mouthwash), creams, pastes, lotions and gels. Topical administration includes application to the oral mucosa, which includes the oral cavity, oral epithelium, palate, gingival, and the nasal mucosa. In some embodiments, the composition contains at least one active component and a suitable vehicle or carrier. It may also contain other components, such as an anti-irritant. The carrier can be a liquid, solid or semi-solid. In embodiments, the composition is an aqueous solution, such as a mouthwash. Alternatively, the composition can be a dispersion, emulsion, gel, lotion or cream vehicle for the various components. In one embodiment, the primary vehicle is water or a biocompatible solvent that is substantially neutral or that has been rendered substantially neutral. The liquid vehicle can include other materials, such as buffers, alcohols, glycerin, and mineral oils with various emulsifiers or dispersing agents as known in the art to obtain the desired pH, consistency and viscosity. It is possible that the compositions can be produced as solids, such as powders or granules. The solids can be applied directly or dissolved in water or a biocompatible solvent prior to use to form a solution that is substantially neutral or that has been rendered substantially neutral and that can then be applied to the target site. In embodiments of the invention, the vehicle for topical application to the skin can include water, buffered solutions, various alcohols, glycols such as glycerin, lipid materials such as fatty acids, mineral oils, phosphoglycerides, collagen, gelatin and silicone based materials.

The composition according to the present invention, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

The dose administered to the mammal, particularly human and other mammals, in accordance with the present invention should be sufficient to affect the desired response. One skilled in the art will recognize that dosage will depend upon a variety of factors, including the age, condition or disease state, predisposition to disease, genetic defect or defects, and body weight of the mammal. The size of the dose will also be determined by the route, timing and frequency of administration as well as the existence, nature, and extent of any adverse side-effects that might accompany the administration of a particular composition and the desired effect. It will be appreciated by one of skill in the art that various conditions or disease states may require prolonged treatment involving multiple administrations.

The composition according to the present invention may be administered in an effective amount. An “effective amount” means an amount sufficient to show a meaningful benefit in an individual, e.g., promoting at least one aspect of tumor cell cytotoxicity (e.g., inhibition of growth, inhibiting survival of a cancer cell, reducing proliferation, reducing size and/or mass of a tumor (e.g., solid tumor)) or anti-viral effect, or treatment, healing, prevention, delay of onset, halting, or amelioration of other relevant medical condition(s) associated with a particular cancer or viral infection. The meaningful benefit observed in the patient can be to any suitable degree (10, 20, 30, 40, 50, 60, 70, 80, 90% or more). In some aspects, one or more symptoms of the cancer or viral infection are prevented, reduced, halted, or eliminated subsequent to administration of a composition according to the present invention, thereby effectively treating the disease to at least some degree.

Effective amounts may vary depending upon the biological effect desired in the individual, condition to be treated, and/or the specific characteristics of the composition according to the present invention and the individual. In this respect, any suitable dose of the composition can be administered to the patient (e.g., human), according to the type of disease to be treated. Various general considerations taken into account in determining the “effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference. The dose of the composition according to the present invention desirably comprises about 0.1 mg per kilogram (kg) of the body weight of the patient (mg/kg) to about 400 mg/kg (for e.g., about 0.75 mg/kg, about 5 mg/kg, about 30 mg/kg, about 75 mg/kg, about 100 mg/kg, about 200 mg/kg, or about 300 mg/kg). In another embodiment, the dose of the composition according to the present invention comprises about 0.5 mg/kg to about 300 mg/kg (for e.g., about 0.75 mg/kg, about 5 mg/kg, about 50 mg/kg, about 100 mg/kg, or about 200 mg/kg), about 10 mg/kg to about 200 mg/kg (for e.g., about 25 mg/kg, about 75 mg/kg, or about 150 mg/kg), or about 50 mg/kg to about 100 mg/kg (for e.g., about 60 mg/kg, about 70 mg/kg, or about 90 mg/kg).

The present disclosure is illustrated and further described in more detail with reference to the following non-limiting examples.

### Examples

### Abbreviations

ADME absorption, distribution, metabolism, and excretion
API Active pharmaceutical ingredients
CNS Central nervous system
DIC N,N′-diisopropylcarbodiimide
DMAP 4-(N,N-dimethylamino)pyridine
DMF Dimethylformamide
DMSO Dimethyl Sulfoxide
EDC 1-ethyl-3-(3-dimethylaminopropyl)carbodiimide
HCQ Hydroxychloroquine
HPLC High performance liquid chromatography
HIV Human immunodeficiency virus
IR Infrared
kg Kilogram
LCMS Liquid Chromatography/Mass Spectrometry
mg Milligram
μg Microgram
mL Milliliter
μL Microliter
mM millimolar
mm Millimeter
mmol Millimole
Mₙ Number average molecular weight
MPS Mononuclear phagocyte system
NMR Nuclear Magnetic Resonance
PDA Photo diode array
PEG Polyethylene glycol
PLS Poly(L-lysine succinylated)
wt Weight

### Example 1. Alexafluor488-labeled Poly(L-Lysine Succinylated) for Scavenger Receptor A1 Targeting

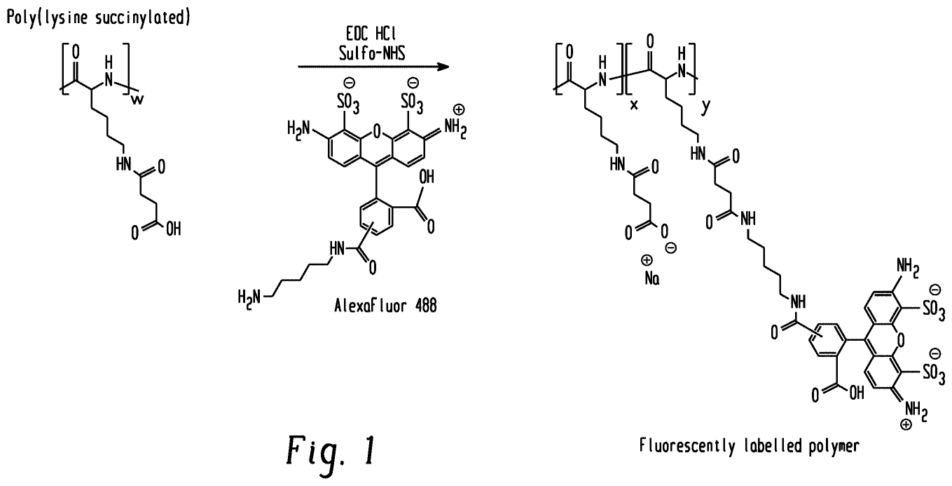

Using EDC (1-ethyl-3-(3-dimethylaminopropyl)carbodiimide) coupling, AlexFluor 488 fluorescent dye was attached to the polymer through stable amide bonds (hereinafter “polymer-488”) (FIG. 1).

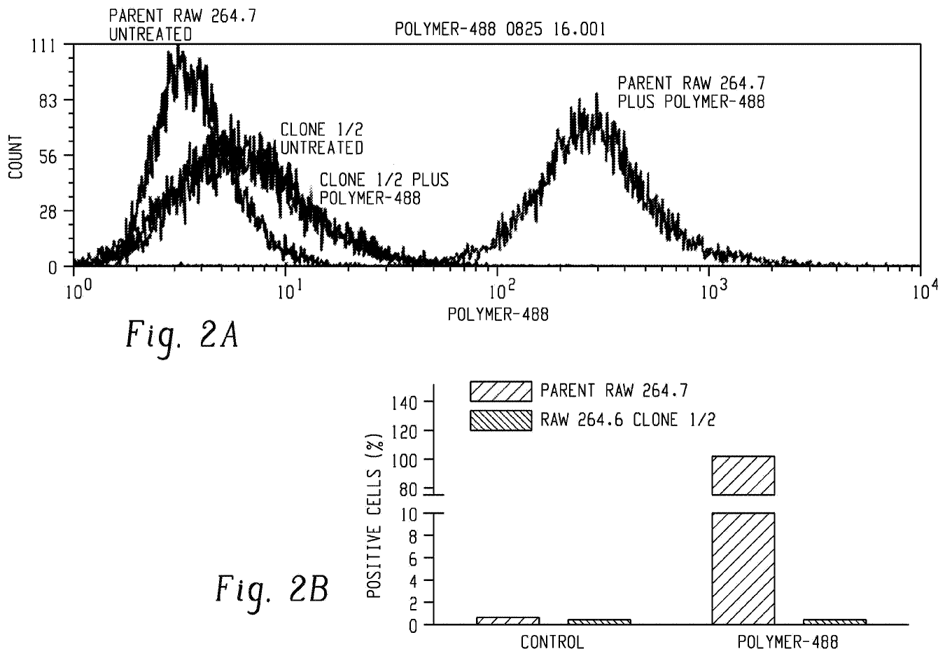

Interactions with scavenger receptor A1 were validated in the Raw 264.7 derivative cells (Clone %) which do not express scavenger receptor A1. Both the parent cells and the SR-A deficient Clone H were treated with the polymer-488 at a concentration of 0.001 mg/ml for 24 hours and analyzed by flow cytometry. Untreated cells were used as control. The flow cytometry results show uptake of the polymer-488 by all cells in the parent Raw 264.7 population and virtually no uptake by the clone H cells (FIG. 2A and FIG. 2B).

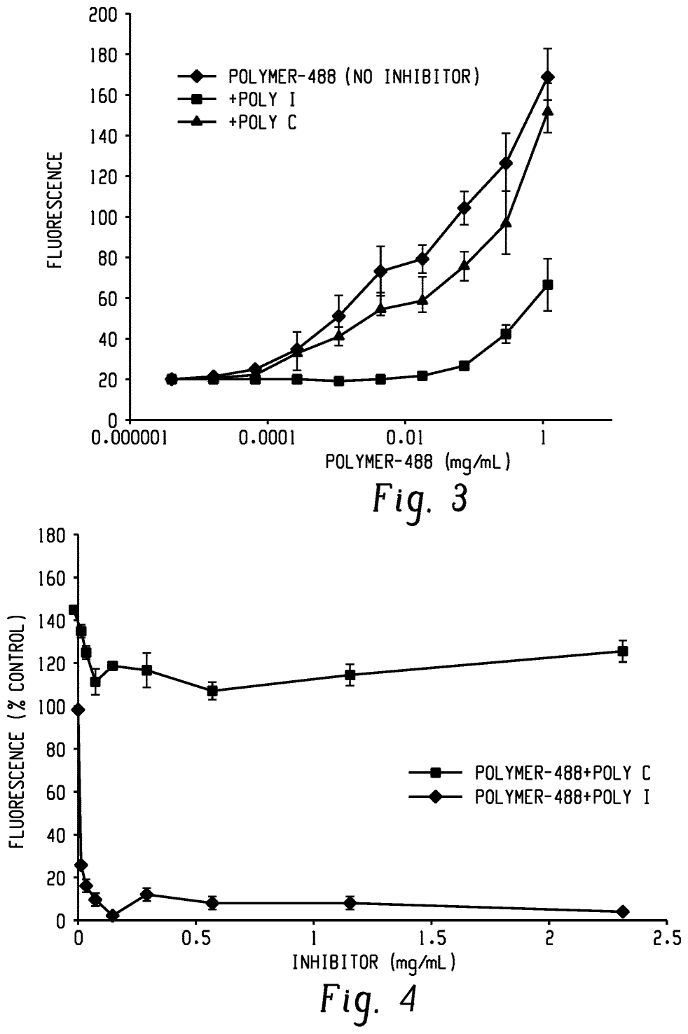

The fluorescently labelled poly(L-lysine succinylated) was also tested for cell uptake in a macrophage cell line Raw 264.7 in the presence of competitive inhibitors and relevant controls. In this experiment, Raw 264.7 macrophages were treated with various concentrations of polymer-488 with either polyinosinic acid (poly I, known inhibitor for scavenger receptor A), polycytidylic acid (poly C, negative control, which does not inhibit scavenger receptor A), or no inhibitor. The results shown in FIG. 3 indicate inhibition of scavenger receptor interaction in presence of poly I and not with poly C, which is consistent with SRA-specific interactions. At higher concentrations of polymer-488, fluorescence is seen due to competing off the poly I.

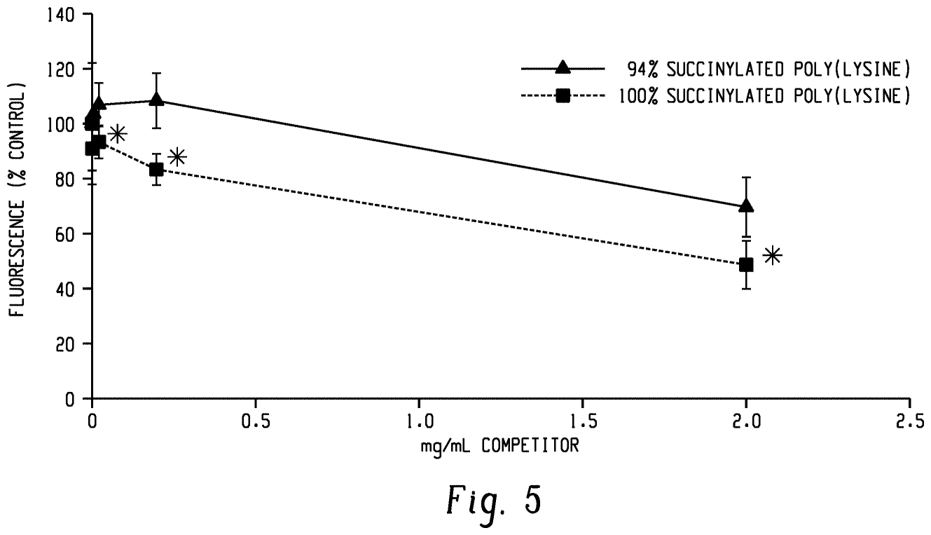

In a follow up study, cells were treated with various concentrations of either poly I or poly C while polymer-488 concentration remained constant. In this study, Raw 264.7 cells were incubated overnight at 400,000 cells/mL. The cells were then treated with various concentrations of polymer-488 and either 200 μg/mL poly I, 200 μg/mL poly C, or no inhibitor. The cells were incubated at 37° C. for 3 hours, washed 3 times with media, and fluorescence measured. The results shown in FIG. 4 indicate virtually no inhibition of fluorescence in the poly C group while there is a dose-dependent inhibition in the poly I treated groups. These results further validate the specific interaction between poly(lysine succinylated) and scavenger receptor A1.

Taken together, the results shown in FIGS. 2-4 indicate a strong interaction between the poly(L-lysine succinylated) and scavenger receptor A1. The polymer appears to have a remarkable ability to be taken up by cells that express receptor A1, and therefore, could be used as a targeted drug delivery system to the cells that express this receptor, particularly macrophages and other myeloid cells.

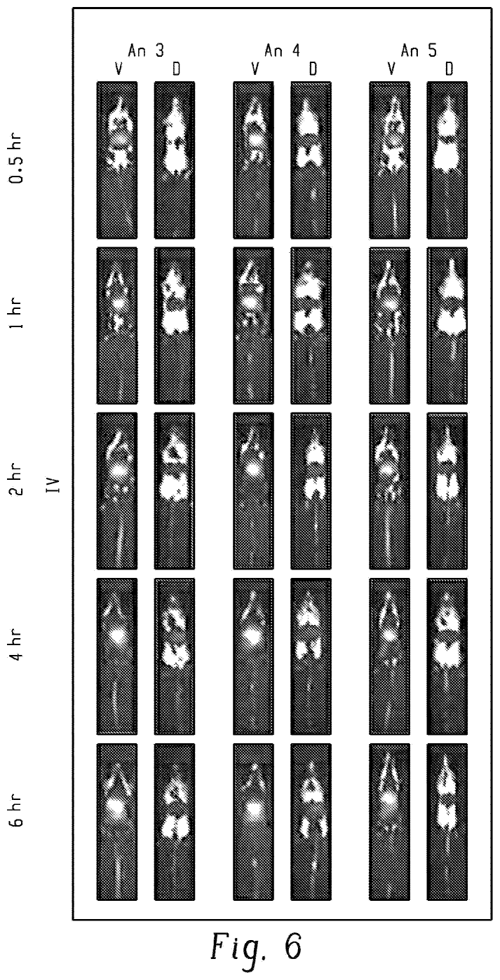

In a follow-up competitive binding study, the binding of poly(L-lysine succinylated), which is 100% succinylated, to Raw 264.7 cells was compared to that of a partially succinylated poly(lysine) to determine if the degree polymer succinylation affects interaction with scavenger receptor A1 on the cell surface. The partially succinylated poly(lysine) was synthesized by reacting poly(L-lysine) (Mₙ=41,000) with succinic anhydride in carbonate buffer followed by acetic anhydride addition. ¹H NMR confirmed the polymer was partially succinylated (94%) and the remaining primary amine groups were capped by acetylation. Raw 264.7 cells were treated with 5 μg/mL fluorescent polymer-488 and various concentrations of fully succinylated (100%) poly(lysine) and partially succinylated (94%) poly(lysine). The cells were incubated at 37° C. for 3 hours, washed 3 times with media, and fluorescence measured. The results displayed in FIG. 5 show dose-dependent decreases in fluorescence when competing with either the 100% or 94% succinylated polymers. Unexpectedly, however, the degree of binding inhibition was dramatically greater for the 100% succinylated polymer in comparison to the 94% succinylated polymer. Since the degree of polymer succinylation was similar for the two constructs (100% vs. 94%), it was unanticipated to see such a large difference in scavenger receptor A1 competition. This unforeseen result shows that even minor differences in the degree of polymer succinylation (i.e., 100% vs. 94%) can have significant effects on receptor interaction, and for this reason, the 100% succinylated poly(lysine) polymer was chosen as the lead prodrug platform over partially succinylated poly(lysine) polymer.

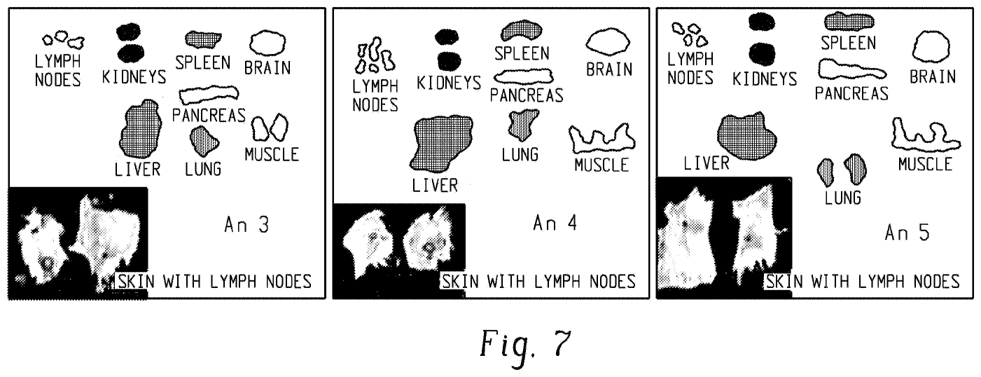

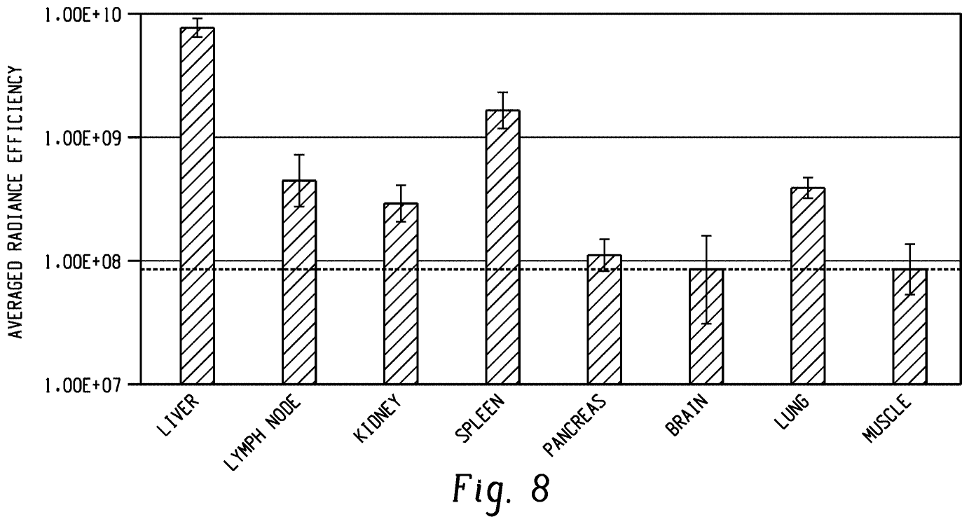

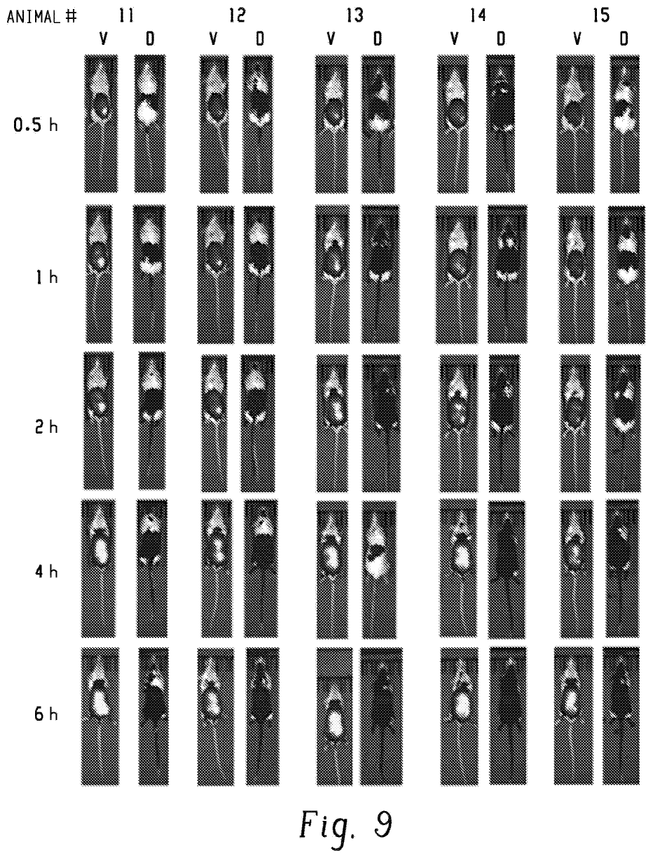

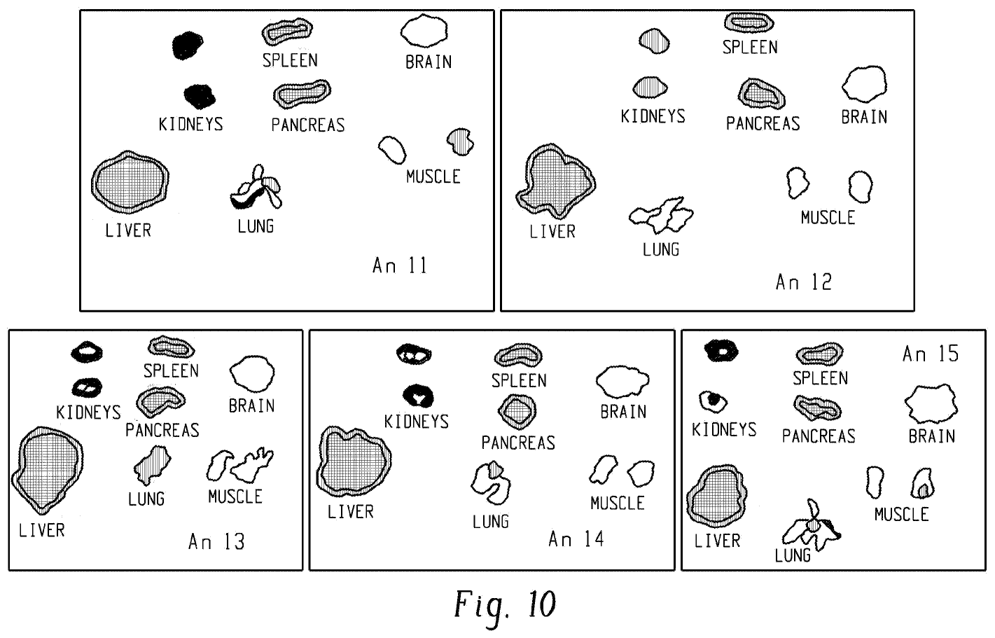

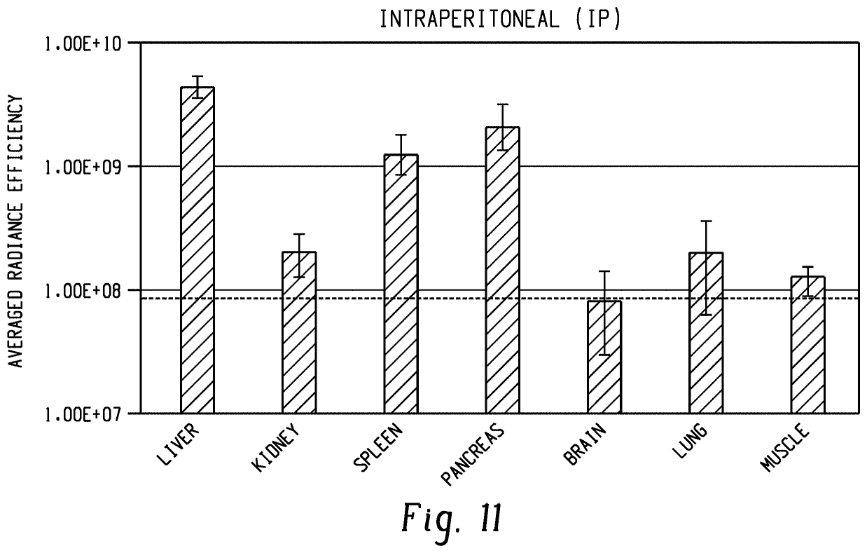

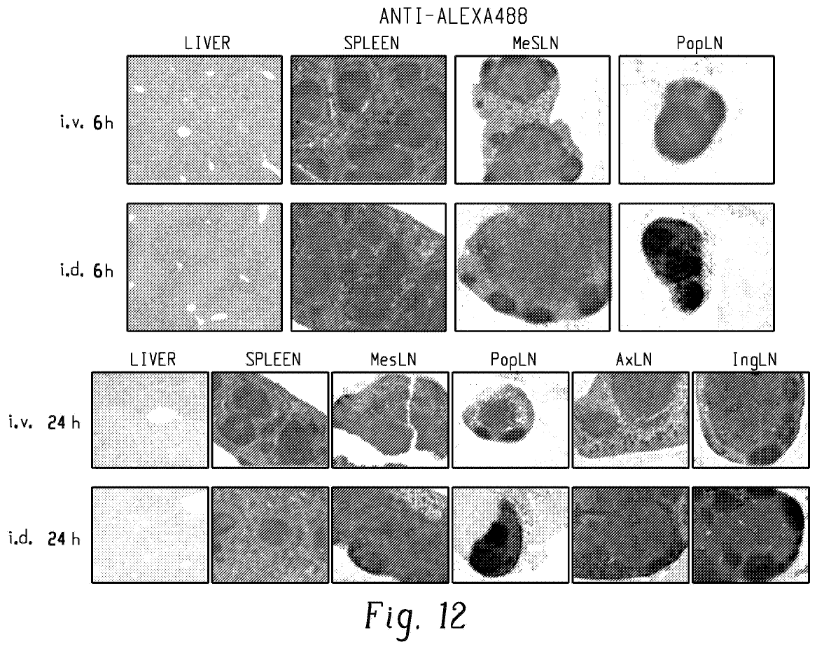

Biodistribution of poly(lysine succinylated) was assessed in mice. The polymer was labelled with Cyanine7.5 amine near-IR dye and administered via tail vein injection or intraperitoneal injection. At various time points, whole-body images were taken, and organs were harvested after 6 hours and imaged. Representative images are shown in FIGS. 6-11. As expected, the polymer is taken up by mononuclear phagocyte system (MPS) organs including liver, spleen, and lung. The polymer is also detected in the lymph node after tail vein injection, which is expected due to the polymer's ability to interact with scavenger receptor A1 on endothelial cells, allowing the polymer to undergo transcytosis into the lymphatic system. This ability of the prodrug to undergo lymphatic translocation following intravenous administration appears to be unique to scavenger receptor A1 ligands, and may have tremendous therapeutic implications for infectious diseases such as HIV. Subcutaneous and intraperitoneal injections also resulted in MPS organ distribution but were not as successful at reaching lymph node, though intraperitoneal injection resulted in distribution to pancreas which may have therapeutic implications for pancreatic cancer.

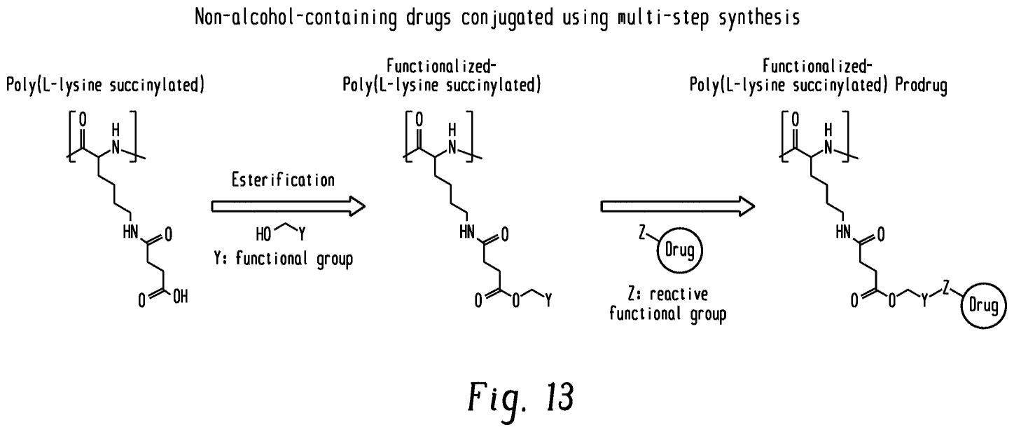

A study of lymph node distribution of the platform was also performed. In this study, mice were injected with polymer-488 via IV or ID administration, and several organs were harvested at 6 and 24 hours. The organs underwent tissue fixation and anti-alexa-488 staining, followed by microscopic imaging. This method allowed for high-resolution imaging of the polymer's distribution in liver, spleen, and various lymph nodes (mesenteric, popliteal, axillary, and inguinal). The resulting images showed accumulation of the prodrug platform in these tissues at both 6 and 24 hour time points (FIG. 12).

### Example 2. Allyl-Functionalized Poly(L-Lysine Succinylated)

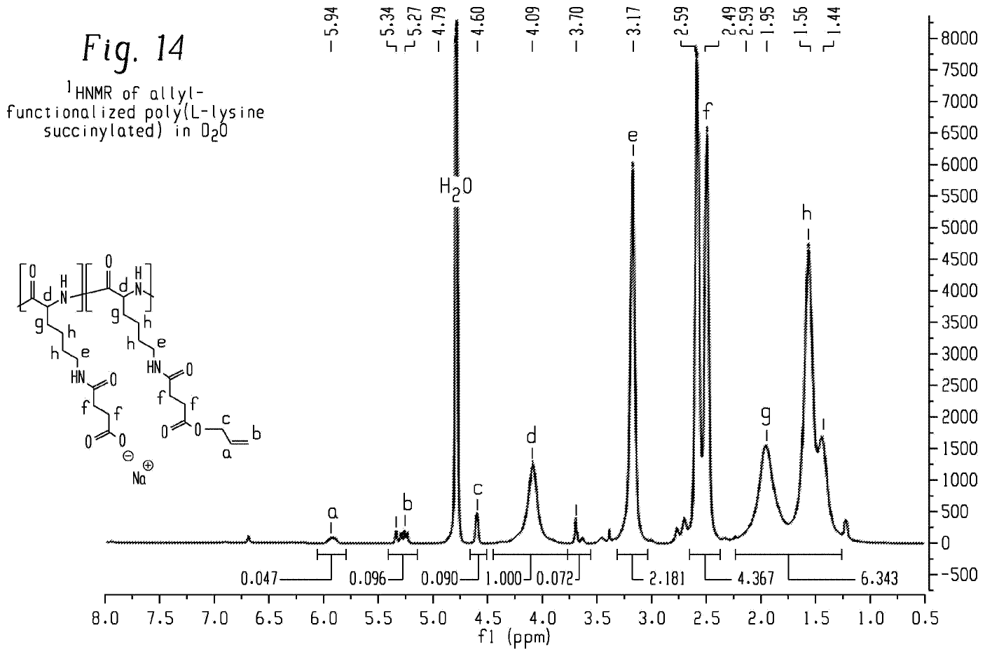

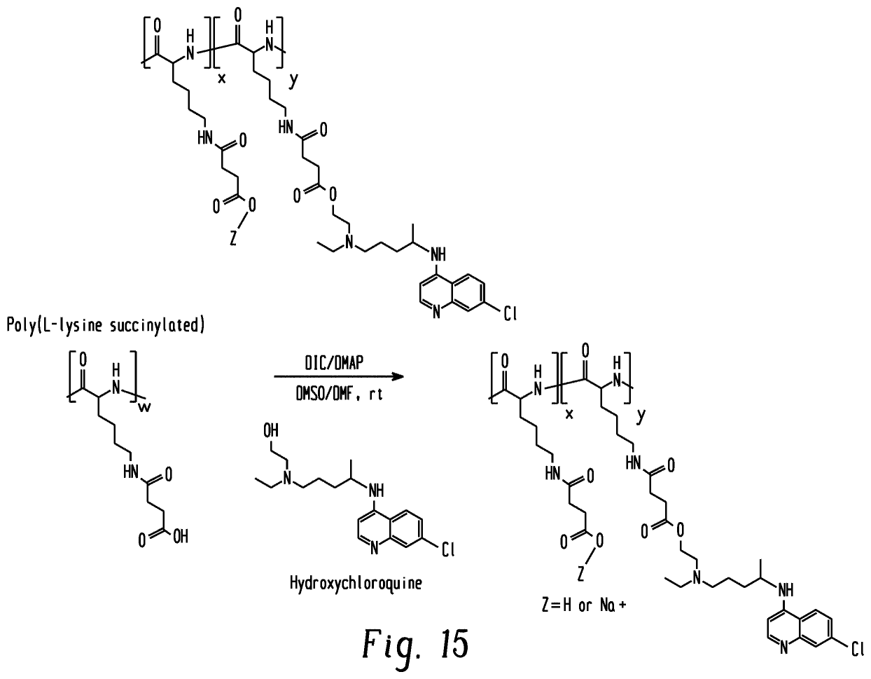

Although drugs containing alcohol groups can be conjugated directly to the polymer using a single-step esterification chemistry, additional synthetic steps are required for drugs lacking a reactive alcohol group. For example, the polymer can be modified with R—OH linkers, where OH is an alcohol that can be conjugated to the polymer using esterification and R is a carbon chain containing a reactive functional group (FIG. 13). Examples of ‘R’ reactive functional groups include alkene, alkyne, azide, thiol, maleimide, aminooxy, ketone, aldehyde, amine, isothiocyanate, and hydrazide. Active pharmaceutical ingredients (APIs), including small molecules, peptides, proteins, oligonucleotides, and other biologics, containing reactive functional groups can be conjugated to the polymer using a specific chemistry. In an example, the poly(L-lysine succinylated) can be modified with allyl alcohol, which can then undergo thiolene chemistry with an API containing a free thiol group. The allyl-functionalized poly(L-lysine succinylated) was synthesized using esterification chemistry described for previous prodrug versions. ¹H NMR analysis confirmed allyl alcohol conjugation, and in this example there was approximately 12 allyl groups per polymer (FIG. 14).

In another example, the poly(L-lysine succinylated) is modified with an alkyne group, which then undergoes alkyne-azide chemistry with an API containing an azide group.

In another example, the poly(L-lysine succinylated) is modified with an azide group, which then undergoes alkyne-azide chemistry with an API containing an alkyne group.

In another example, the poly(L-lysine succinylated) is modified with a thiol group, which then undergoes thiolene chemistry with an API containing an alkene or maleimide group.

In another example, the poly(L-lysine succinylated) is modified with a maleimide group, which then undergoes thiolene chemistry with an API containing a free thiol group.

In another example, the poly(L-lysine succinylated) is modified with an aminooxy group, which then reacts with an API containing an aldehyde or ketone group to form an oxime bond.

In another example, the poly(L-lysine succinylated) is modified with a ketone group, which then reacts with an API containing an aminooxy group.

In another example, the poly(L-lysine succinylated) is modified with an aldehyde group, which then reacts with an API containing a hydrazide or aminooxy group.

In another example, the poly(L-lysine succinylated) is modified with an amine group, which then reacts with an API containing an isothiocyanate or NHS-ester group.

In another example, the poly(L-lysine succinylated) is modified with an isothiocyanate group, which then reacts with an API containing an amine group.

In another example, the poly(L-lysine succinylated) is modified with a hydrazide group, which then reacts with an API containing an aldehyde group.

The following Examples illustrate the synthesis and characterization of Poly(L-lysine succinylated)-hydroxychloroquine

### Example 3. Synthesis of 10 k and 25 k Hydroxychloroquine Poly(L-lysine succinylated) Prodrug

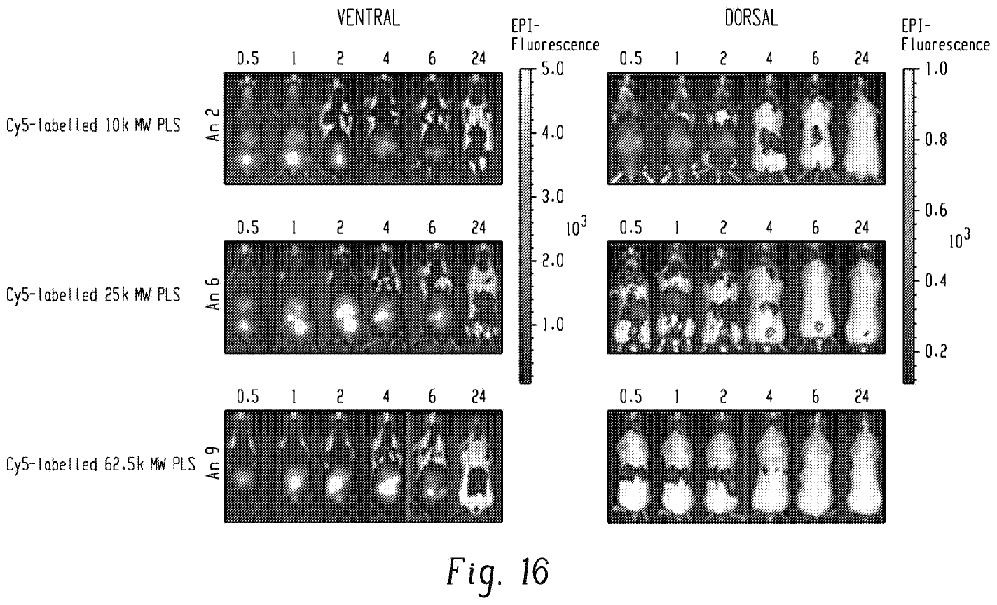

Hydroxychloroquine was selected as a model anti-parasitic drug. Using the carbodiimide chemistry below, hydroxychloroquine was conjugated to the poly(L-lysine succinylated) by an ester bond (FIG. 15).

Poly(L-lysine succinylated) (PLS) (degree of polymerization: 40, molecular weight: 10,000 g/mol) was converted to its free acid form by dissolving 500 mg polymer in 10 mL water, acidifying with 2.4 mL IN HCl, centrifuging at 5,000×g for 2 min to form a pellet, rinsing the pellet several times with water, and lyophilizing. The free acid form of PLS-40 (300 mg, 1.32 mmol acid) was added to hydroxychloroquine (HCQ; 66.3 milligram (mg), 0.197 mmol) in an oven-dried 50-mL round bottom flask equipped with stir bar. The flask was capped with a rubber septum and purged with nitrogen for 5 minutes. Anhydrous DMF (7.5 mL) was added to the reaction flask, followed by bath sonication at 37° C. until the polymer and drug were completely dissolved. Separately, 4-dimethylaminopyridine (DMAP) was weighed into an oven-dried vial, capped with rubber septum, and purged with nitrogen for 5 minutes. The DMAP was dissolved using anhydrous DMSO to provide a DMAP concentration of 54 mg/mL. The DMAP solution (3.00 mL, 1.32 mmol DMAP) was added to the reaction flask via syringe. N,N′-diisopropylcarbodiimide (DIC; 122 μL, 0.789 mmol) was added to the reaction flask via syringe. The reaction was allowed to stir at room temperature for 5 hours. The reaction was quenched with 10 mL sodium acetate buffer (100 mM, pH 5.8) and 10 mL acetone. The product was purified using dialysis tubing (molecular weight cutoff of 2 kDa) in different solvents in the following order: 100% acetone→50% acetone in water→sodium acetate buffer, pH 5.8→100% water. The pH inside the dialysis tubing was then raised to 6.3 and underwent several rounds of dialysis against 100% water. The product was sterile filtered and lyophilized to yield a fluffy, white material.

### Example 4. Synthesis of 25 k Hydroxychloroquine Poly(L-lysine succinylated) Prodrug

This prodrug was synthesized using an identical method described above, except using Poly(L-lysine succinylated) (Degree of polymerization: 100, molecular weight: 25,000 g/mol). All other details were unchanged.

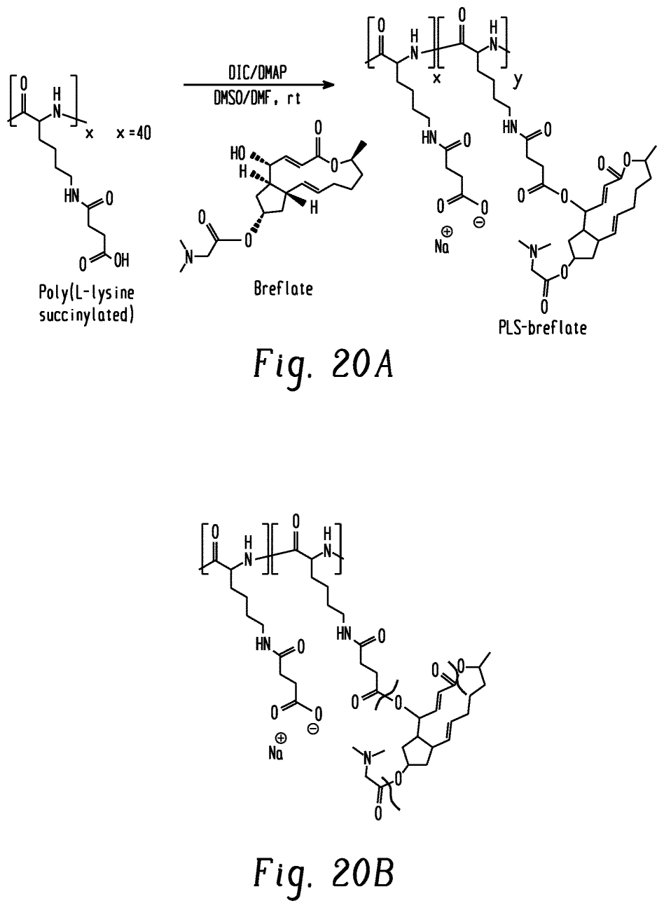

Drug loading of HCQ in PLS-HCQ prodrugs was determined using a sodium hydroxide (NaOH) hydrolysis method followed by HPLC/UV analysis. PLS-HCQ samples were prepared at 1.00 mg/mL and HCQ standards at 12.5, 25.0, 50.0, 100, and 200 μg/mL in 20% acetonitrile in water (FIGS. 19A and 19B). 150 microliter (μL) of each sample was treated with 10 μL 1 normal (N) NaOH for 1 hour (h) to completely hydrolyze all esters, followed by neutralization with 10 μL IN HCl. A set of non-degraded HCQ standards were prepared by taking 150 μL of each standard and adding 20 μL water. The samples were analyzed by an HPLC/PDA system consisting of an LC-20AT pump, SIL-20AC auto injector, CTO-20AC column oven, SPD-M20A diode array detector, and C-R3A integrator (Shimadzu Scientific Instruments, Inc.). The HPLC conditions for the NaOH-treated samples were: 10 μL injection volume with an isocratic elution using 15% acetonitrile (0.1% formic acid) in water (0.1% formic acid). Flow rate was 1.00 milliliters per minute (mL/min), and column temperature was 35° C. PDA monitored at 330 nanometers (nm). The column used was Zorbax SB C18 4.6 millimeters (mm) id x 15 centimeters (cm). As shown in Table 1, the drug loading as high as about 18% HCQ (weight to weight) has been achieved.

TABLE 1
Drug loading analysis of PLS-HCQ prodrugs

| Sample | Drug loading (% wt/wt) |
|---|---|
| 25k PLS-HCQ | 17.5 ± 0.7 |
| 10k PLS-HCQ | 18.0 ± 1.6 |

Data presented as mean ± SD (n = 3)

### Example 5. Size-Dependent Brain and Lymphatic Distribution of Poly(L-Lysine Succinylated) Polymer

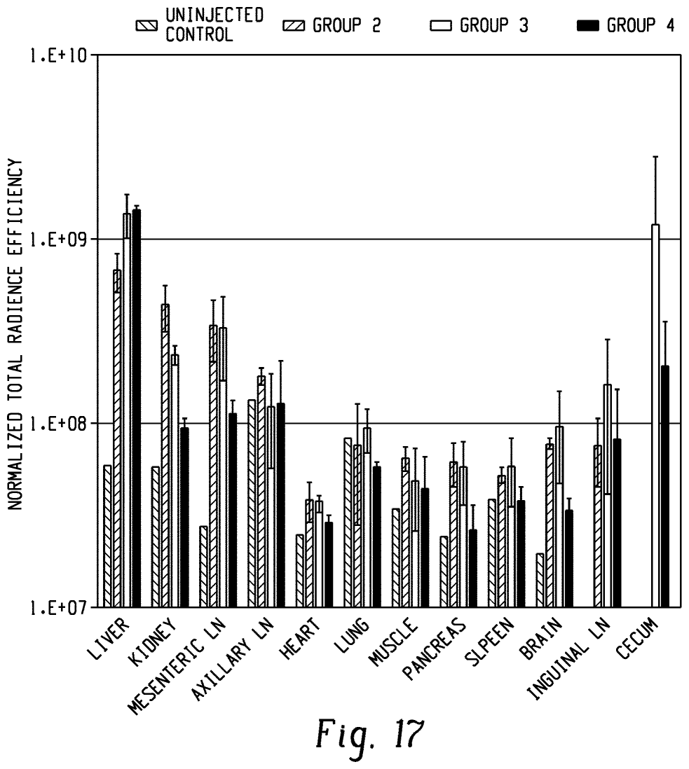

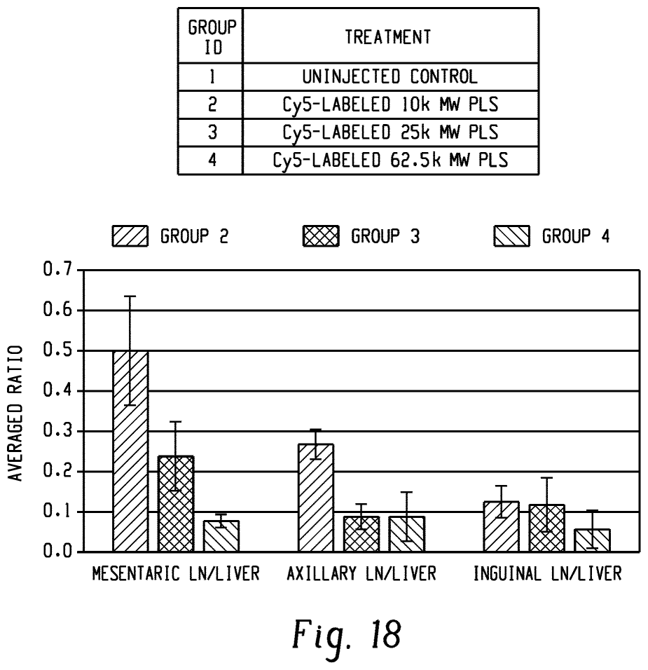

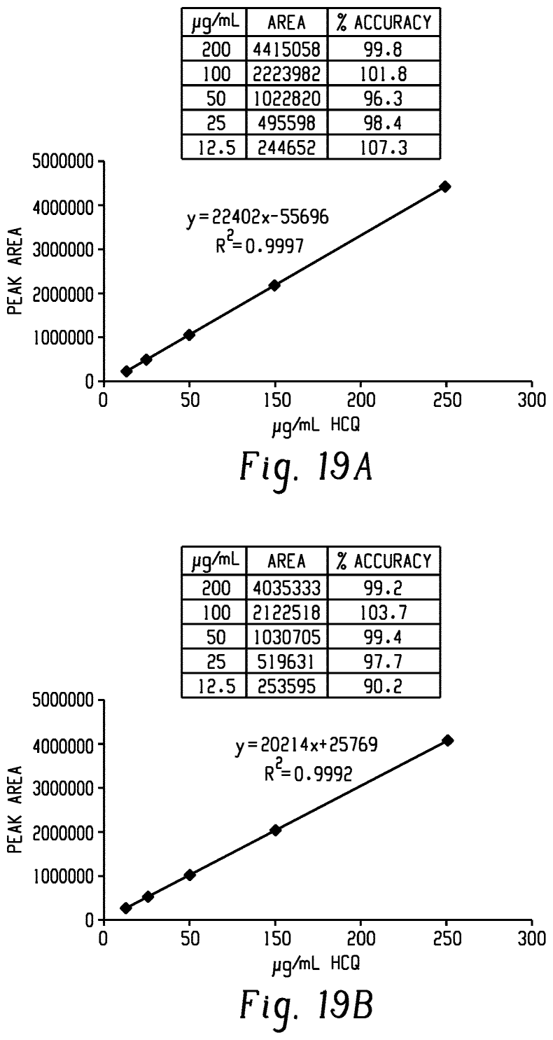

Biodistribution of Poly(L-lysine succinylated) polymers of different sizes (10 kDa, 25 kDa, 62.5 kDa) were assessed in mice. The polymers were labelled with Cyanine5 amine near-IR dye and administered via tail vein injection. At various time points, whole-body images were taken, and organs were harvested after 6 hours and imaged. Representative images are shown in FIGS. 16-18. As expected, the polymer is taken up by mononuclear phagocyte system (MPS) organs including liver, spleen, and lung. The polymer is also detected in the lymph node and brain after tail vein injection, which is expected due to the polymer's ability to interact with scavenger receptor A1 on endothelial cells, allowing the polymer to undergo transcytosis into the lymphatic and CNS systems. This ability of the prodrug to undergo lymphatic translocation following intravenous administration appears to be unique to scavenger receptor A1 ligands, and may have tremendous therapeutic implications for infectious diseases (such as HIV). Remarkably, lymphatic and brain distribution was size-dependent, with the smaller 10 k and 25 k polymers having greater lymphatic and brain distribution than the larger 65 k polymer. This is an unexpected finding, as the previous literature has reported that larger succinylated proteins, with more anionic groups, have higher brain endothelial transcytosis (Tokuda H., Masuda S., Takakura Y., Sezaki H., Hashida M., Specific uptake of succinylated proteins via a scavenger receptor-mediated mechanism in cultured brain microvessel endothelial cells. Biochem Biophys Res Commun. 1993, 196 (1):18-24; Tokuda, H.; Nagano, H.; Takakura, Y.; Hashida, M., Uptake Characteristics of Anionized and Cationized Proteins in Brain Microvessel Endothelial Cells. Pharmaceutical Sciences 1997, 3: 147-151). This is also unexpected since the smaller 10 k and 25 k polymers are below the molecular weight cut-off for renal clearance, and rapidly eliminated from the circulation via renal clearance as evidenced by the kidney and bladder fluorescence observed at early time points for the Cy5 labeled 10 k and 25 k PLS in our studies (Jorgensen, K. E.; Moller, J. V., Use of Flexible Polymers as Probes of Glomerular Pore-Size. Am J Physiol 1979, 236 (2), F103-F111.)

The following Examples illustrate the synthesis and characterization of Poly(L-lysine succinylated) breflate Prodrug (PLS-breflate).

### Example 6. Synthesis of PLS-breflate

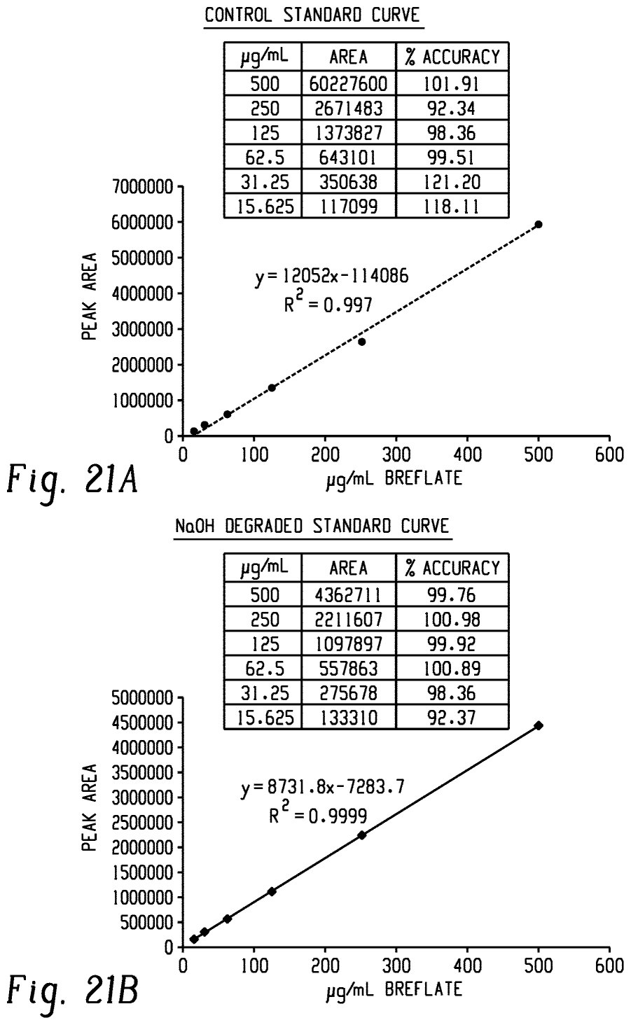

Breflate was conjugated to the pendant carboxyl groups of PLS using 4-dimethylaminopyridine (DMAP)-catalyzed carbodiimide esterification (FIG. 20A). Briefly, poly(L-lysine succinylated) 10KDa (PLS-10) was converted to the free acid form by dissolving 2000 mg PLS-10 into an 80 mL cold water and adding 4.4 mL of 1 N HCl. The resulting precipitant (PLS-10-COOH) was pelleted by centrifugation, washed several times with water, and lyophilized (˜1850 mg yield). PLS-10-COOH (1800 mg) and breflate (432 mg) were weighed and added to an oven-dried 100 mL round-bottom flask equipped with a stir bar. The flask was capped with a rubber septum and purged with nitrogen for 5 min. Anhydrous DMF (45 mL) was added to the flask followed by sonication until dissolution was completed. In an oven-dried vial, DMAP (1158 mg) was added, and the vial was capped and purged with nitrogen for 5 min. The DMAP was then dissolved with 18 mL anhydrous DMSO under nitrogen. The DMAP solution was transferred to the PLS-COOH/breflate reaction flask under nitrogen via a syringe. DIC (734 μL) was added to the reaction flask dropwise via a microsyringe, and the reaction was allowed to stir at room temperature. The reaction was monitored using high-performance liquid chromatography (HPLC) for approximately 5 h until unreacted breflate was undetectable. The reaction was then diluted with a 100 mM sodium acetate buffer (pH 5.8) and dialyzed in a Spectra/P or 6 regenerated cellulose dialysis tubing (3 k molecular weight cutoff) against acetone overnight. To completely remove the DMAP without cleaving the polymer prodrug product, dialysis proceeded in different solvents in the following order: 50% acetone in water→sodium acetate buffer pH 5.8→100% water. Next, the product was converted to the sodium salt by increasing the pH inside the dialysis bags above 6 using saturated sodium bicarbonate solution. Several rounds of dialysis against 100% water were performed at 4° C. to remove bicarbonate salts. Finally, the product was sterile-filtered and lyophilized to yield a fluffy, white material (1600 mg).

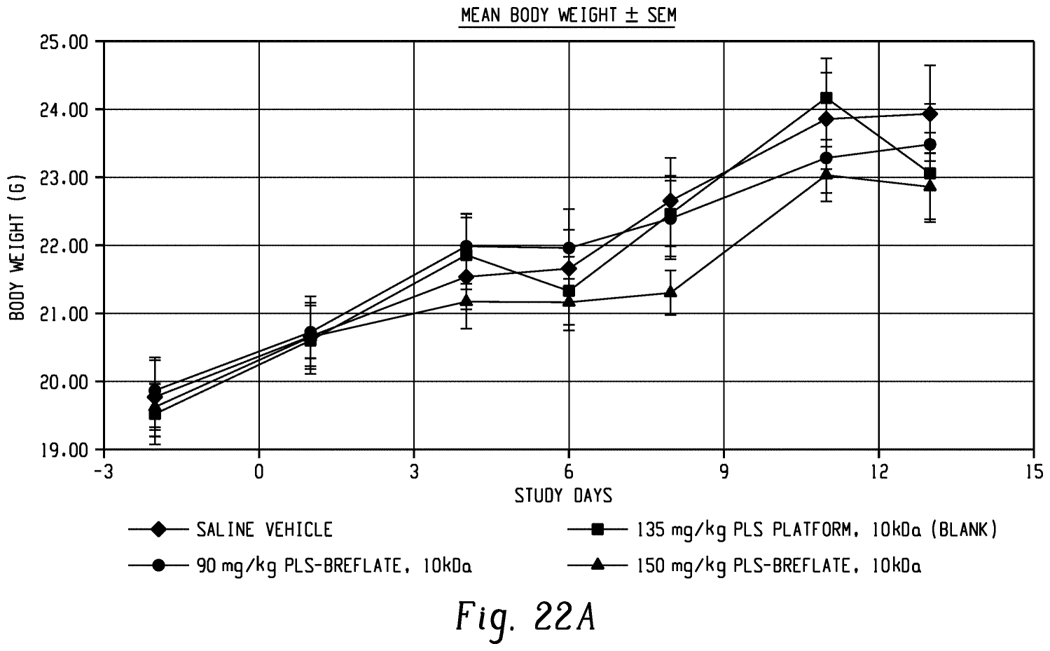

Drug loading determination, as a weight percent, was performed using a sodium hydroxide (NaOH) degradation method followed by HPLC analysis. Prodrug samples were treated with 1 N NaOH for 1 h to completely hydrolyze the linker ester and release all of the breflate from the prodrug (FIG. 20B). A standard curve of NaOH-treated breflate was generated (FIG. 21B) and used to quantify the breflate content in PLS-breflate after NaOH treatment (FIG. 21A). Excellent breflate loading at 16.7% (weight by weight (wt/wt)) (Table 2) was achieved. No free breflate or other contaminants in the prodrug sample were detected by HPLC.

TABLE 2
Percent weight of breflate in prodrug sample analyzed by
HPLC (NaOH treated breflate standards)

| Degraded | area | ug/mL | % wt | % wt (AVG) | SD |
|---|---|---|---|---|---|
| PLS-breflate | 1475199 | 168.11 | 16.81 | | |
| PLS-breflate | 1439420 | 164.01 | 16.40 | | |
| PLS-breflate | 1486817 | 169.44 | 16.94 | 16.72 | 0.28 |

### Example 7. PLS-Breflate Efficacy Study in the LOX IMVI Mouse Model Study Design

LOX IMVI human melanoma cancer cells were implanted subcutaneously into the left flank of female athymic nude mice, aged 7-9 weeks (Charles River Laboratories, Inc., Wilmington, Mass.), at 5×10⁶ cells per 100 uL of HBSS (Hanks Balanced Salt Solution). Before initiation of treatment, animals were randomized by tumor volume using StudyLog Software. Treatment groups consisted of a saline control, blank PLS platform, and two dose levels of PLS-breflate (90 and 150 mg/kg). All animals were dosed intraperitoneally (i.p.) at a volume of 0.75 mL/10 g of body weight when tumors reached ˜63 mm³ in volume (3 days post-implantation). The dosing regimen was once every 3 days for 3 total injection (q3d×3). See Table 3 for efficacy study design summary.

All animals were monitored daily for mortality and signs of pharmacologic or toxicologic effects. Animal body weights and tumor volumes were measured and recorded every alternate weekday (M, W, F). Animals showing signs of morbidity, ≥20% loss in body weight, or inability to obtain food or water were euthanized by CO₂ asphyxiation. Animals were also euthanized via CO₂ asphyxiation when/if tumor size reached approximately two centimeters in diameter or if the tumor became ulcerated.

TABLE 3
PLS-breflate Efficacy Study Design: Details of the efficacy study design, ADME Tox
198

| | | | | | | | |
|---|---|---|---|---|---|---|---|
| | # OF | ANIMAL | SEX | Dose | | | Dosing |
| TREATMENT | ANIMALS | #'S | (M/F) | Level | VEHICLE | ROUTE | schedule |

Athymic nu/nu mice

| Disease induction: Injection of 5 × 10⁶ LOX IMVI (human | | | | 100 uL | HBSS | s.c. | — |
|---|---|---|---|---|---|---|---|
| melanoma cancer cell line) | | | | | | flank | |
| Saline vehicle | 10 | 05, 11, | F | 0.75 | Saline | i.p. | Q3dx3; 3 |
| | | 14, 21, | | mL/10 g | | | injections |
| | | 26, 27, | | of BW | | | |
| | | 43, 46, | | | | | |
| | | 57, 63 | | | | | |
| 135 mg/kg PLS platform, | 10 | 02, 03, | F | 0.75 | Saline | i.p. | Q3dx3; 3 |
| 10 kDa (blank) | | 04, 12, | | mL/10 g | | | injections |
| | | 17, 28, | | of BW | | | |
| | | 32, 33, | | | | | |
| | | 61, 64 | | | | | |
| 90 mg/kg PLS-breflate, | 10 | 22, 24, | F | 0.75 | Saline | i.p. | Q3dx3; 3 |
| 10 kDa | | 38, 40, | | mL/10 g | | | injections |
| | | 54, 55, | | of BW | | | |
| | | 70, 74, | | | | | |
| | | 76, 77 | | | | | |
| 150 mg/kg PLS-breflate, | 10 | 08, 09, | F | 0.75 | Saline | i.p. | Q3dx3; 3 |
| 10 kDa | | 16, 39, | | mL/10 g | | | injections |
| | | 45, 47, | | of BW | | | |
| | | 48, 59, | | | | | |
| | | 62, 75 | | | | | |

### In-Life Results

### Body Weight

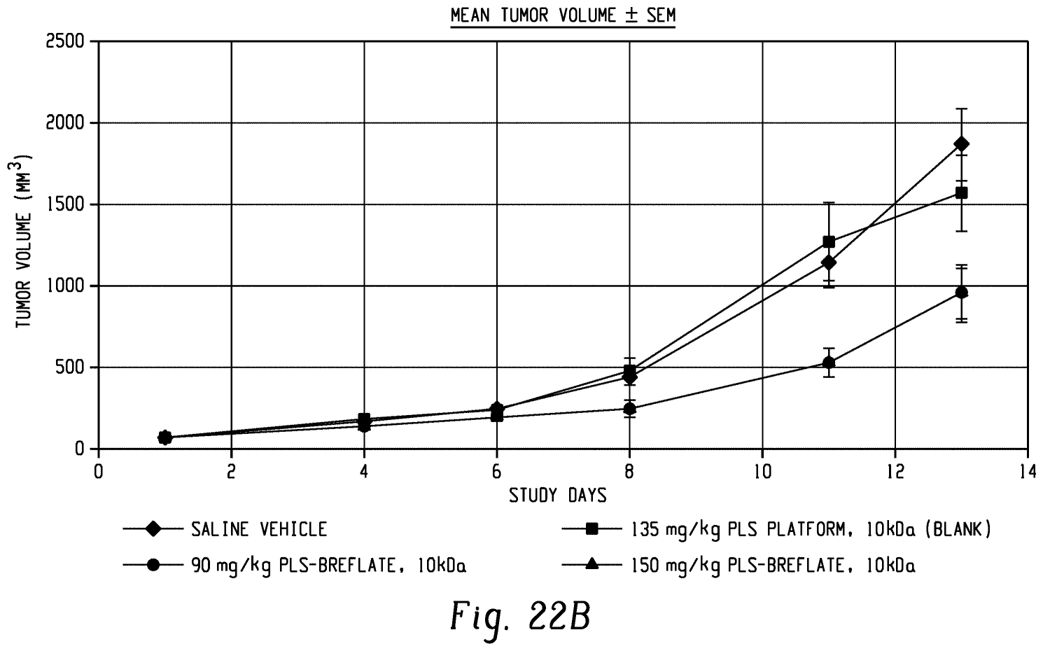

No significant changes in body weight were observed throughout the duration of the study, and no animals were terminated due to excessive body weight loss. FIG. 22A displays the average body weight for each group throughout the duration of the study.

### Tumor Volume

Non-dose dependent suppression of tumor growth is associated with administration of PLS-breflate. Significant differences in tumor volume for PLS-breflate treated animals in comparison to saline and blank PLS control treated animals were observed from study day 8 onwards. FIG. 22B displays the tumor growth for saline control, PLS platform (blank), and PLS-breflate prodrug treatment groups throughout the duration of the study.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the present detailed description as defined by the following claims.

1. A method for delivery of a therapeutically active molecule to the brain or lymphatics of a patient, the method comprising the steps of:
providing a composition comprising a conjugate of completely succinylated poly(lysine succinylated) polymer and a therapeutically active molecule, and
administering the composition to a patient,
wherein said succinylated poly(lysine succinylated) polymer has a molecular weight between 10,000 grams per mole and 25,000 grams per mole,
wherein said conjugate contains free/unmodified succinyl groups available for scavenger receptor A1-targeting;
wherein the conjugate targets scavenger receptor A1;
wherein the conjugate is represented by the following formula 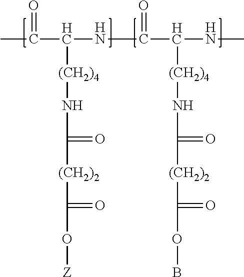
wherein Z=H or Na⁺ and B is a portion of the therapeutically active molecule, and
wherein the therapeutically active molecule is represented by a general formula B—X, wherein X is OH or NH₂;
wherein the therapeutically active molecule is present in a physiologically effective amount; and
wherein said composition targets the brain or lymphatics of said patient.

2. (canceled)

3. The method of claim 1, wherein the therapeutically active molecule is a small molecule drug, a peptide, or a vaccine.

4. The method of claim 1, wherein the therapeutically active molecule is an anti-cancer drug, an immunotherapy drug, an anti-viral drug, an anti-parasitic drug, a CNS drug, or an antibacterial.

5. The method of claim 4,
wherein the anti-cancer drug is gemcitabine, rapamycin, PI-103, PF-04691502, AZD-8055, torkinib, KU-0063794, PX-886, apitolisib, everolimus, hydroxychloroquine, resiquimod, galactosyl ceramide, or breflate,
wherein anti-viral drug is abacavir, atazanavir, everolimus, lamivudine, emtricitabine, lopinavir, rapamycin, ritonavir, tenofovir, dolutegravir, zidovudine, hydroxychloroquine, or ribavirin;
wherein the anti-parasitic drug is hydroxychloroquine, quinine, mefloquine, doxycycline, atovaquone, metronidazole, or ivermectin;
wherein the CNS drug is, valproate, haloperidol, galantamine, gabapentin, rotigotine or trehalose; or
wherein the antibacterial drug is rifampicin.

6-9. (canceled)

10. The method of claim 1, wherein the amount of the therapeutically active molecule is about 1% to about 20% by weight based on the total weight of the conjugate.

11. The method of claim 1, wherein a number of the therapeutically active molecules conjugated per molecule of completely succinylated poly(lysine succinylated) is about 1 to about 30.

12. The method of claim 1, wherein the amount of the therapeutically active molecule is greater than about 40% by weight based on the total weight of the conjugate.

13. The method of claim 1, wherein a number of the therapeutically active molecules conjugated per molecule of poly(lysine succinylated) is greater than about 30.

14. A composition for delivery of a therapeutically active molecule to the brain or lymphatics of a patient, the composition comprising a conjugate of completely succinylated poly(lysine succinylated) and a therapeutically active molecule;
wherein the conjugate has a molecular weight between 10,000 and 25,000 grams per mole or greater;
wherein the conjugate contains free/unmodified succinyl groups available for scavenger receptor A1-targeting;
wherein the conjugate targets scavenger receptor A1;
wherein the conjugate is represented by the following formula 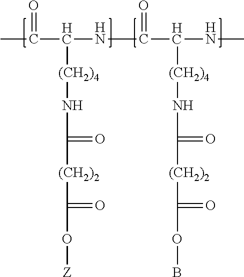
wherein Z=H or Na⁺ and B is a portion of the therapeutically active molecule,
wherein the therapeutically active molecule is represented by a general formula B—X, wherein X is OH or NH₂; and
wherein said composition targets the brain and lymphatics of said patient.

15. The composition of claim 14, wherein the therapeutically active molecule is a vaccine, an anti-cancer drug, an immunotherapy drug, an anti-viral drug, an anti-parasitic drug, a CNS drug, or an antibiotic.

16. The composition of claim 15,
wherein the anti-cancer drug is gemcitabine, rapamycin, PI-103, PF-04691502, AZD-8055, torkinib, KU-0063794, PX-886, apitolisib, everolimus, hydroxychloroquine, resiquimod, galactosyl ceramide or breflate;
wherein the anti-viral drug is abacavir, atazanavir emtricitabine everolimus lamivudine, lopinavir, rapamycin, ritonavir, tenofovir, ribavarin or zidovudine;
wherein the anti-parasitic drug is hydroxychloroquine, quinine, mefloquine, doxycycline, atovaquone, metronidazole, or ivermectin;
wherein the CNS drug is, valproate, haloperidol, galantamine, gabapentin, rotigotine or trehalose; or
wherein the antibacterial drug is rifampicin.

17. The composition of claim 16, wherein the conjugate is of a formula selected from the group consisting of: 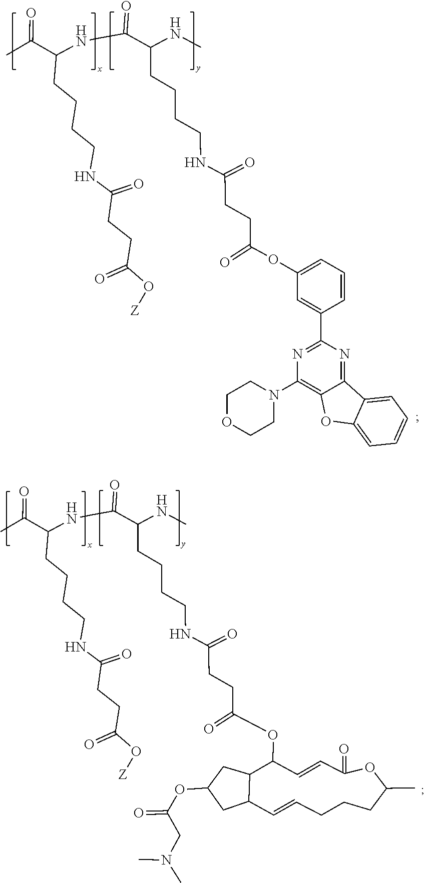 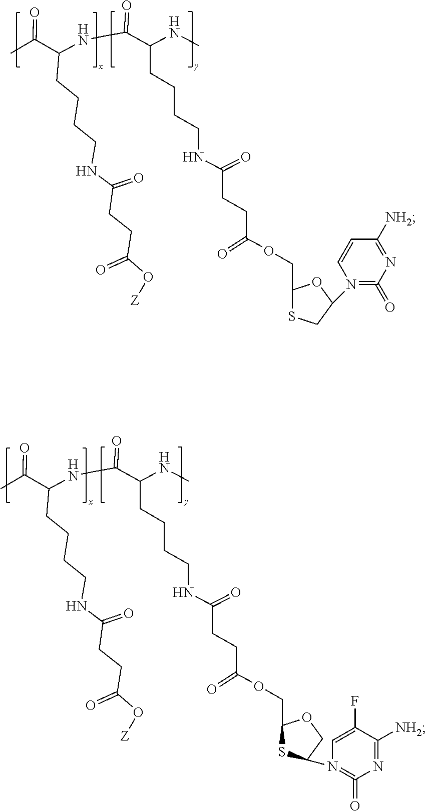 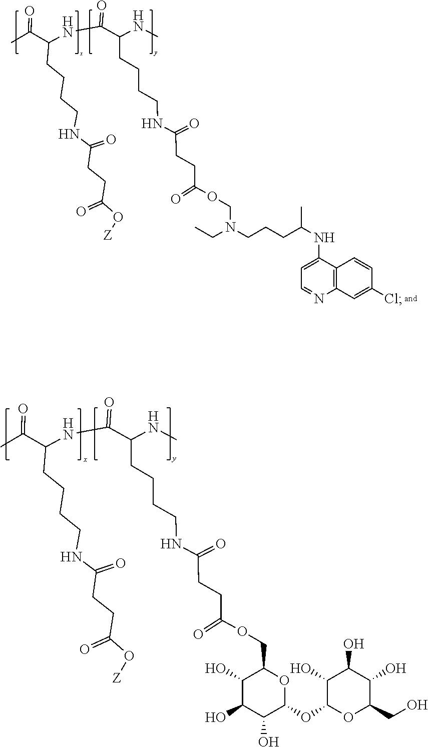
and wherein, in the above formulas,
x and y are variable selected such that x+y=1, and
Z is H or Na.

18-26. (canceled)

27. The composition of claim 14, wherein the therapeutically active molecule does not include paclitaxel.

28. The composition of claim 14, wherein the amount of completely succinylated poly(lysine succinylated) is about 85% based on the total weight of the conjugate.

29. The composition of claim 14, wherein the amount of the therapeutically active molecule is about 15% based on the total weight of the conjugate.

30. The composition of claim 14, wherein a number of the therapeutically active molecules conjugated per molecule of poly(lysine succinylated) is about 10 to about 50.

31. The composition of claim 14, wherein the composition further comprises at least one pharmaceutically acceptable excipient selected from a disintegrator, a binder, a filler, and a lubricant.

32. The composition of claim 31,
wherein the disintegrator is selected from agar-agar, algins, calcium carbonate, carboxymethylcellulose, cellulose, clays, colloid silicon dioxide, croscarmellose sodium, crospovidone, gums, magnesium aluminium silicate, methylcellulose, polacrilin potassium, sodium alginate, low substituted hydroxypropylcellulose, and cross-linked polyvinylpyrrolidone hydroxypropylcellulose, sodium starch glycolate, and starch,
wherein the binder is selected from microcrystalline cellulose, hydroxymethyl cellulose, and hydroxypropylcellulose;
wherein the filler is selected from calcium carbonate, calcium phosphate, dibasic calcium phosphate, tribasic calcium sulfate, calcium carboxymethylcellulose, cellulose, dextrin derivatives, dextrin, dextrose, fructose, lactitol, lactose, magnesium carbonate, magnesium oxide, maltitol, maltodextrins, maltose, sorbitol, starch, sucrose, sugar, and xylitol; or
wherein the lubricant is selected from agar, calcium stearate, ethyl oleate, ethyl laureate, glycerin, glyceryl palmitostearate, hydrogenated vegetable oil, magnesium oxide, magnesium stearate, mannitol, poloxamer, glycols, sodium benzoate, sodium lauryl sulfate, sodium stearyl, sorbitol, stearic acid, talc, and zinc stearate.

33-35. (canceled)

36. The composition of claim 14, wherein the amount of the therapeutically active molecule is about 1% to about 20% by weight based on the total weight of the conjugate.

37. The composition of claim 14, wherein a number of the therapeutically active molecules conjugated per molecule of poly(lysine succinylated) is about 1 to about 30.

38. The composition of claim 14, wherein the amount of the therapeutically active molecule is greater than about 40% by weight based on the total weight of the conjugate.

39. The composition of claim 14, wherein a number of the therapeutically active molecules conjugated per molecule of poly(lysine succinylated) is greater than about 30.
